(19) United States
(12) Patent Application Publication
(10) Pub. No.: US 2026/0335678 A1
(43) Pub. Date: Oct. 29, 2026

(54) RADIATION DELIVERY DETERMINATION SYSTEMS AND METHODS

(71) Applicant: BARD PERIPHERAL VASCULAR, INC., Franklin Lakes, NJ (US)

(72) Inventors: Christopher D. DROBNIK, Wauconda, IL (US); Brandon SIMMONS, Tempe, AZ (US); Amanda THYSTRUP, Phoenix, AZ (US); Mark Nicholas WRIGHT, Coventry, RI (US)

(73) Assignee: BARD PERIPHERAL VASCULAR, INC., Franklin Lakes, NJ (US)

(21) Appl. No.: 18/997,479

(22) PCT Filed: Aug. 5, 2022
(86) PCT No.: PCT/US2022/039511

§ 371 Date: Jan. 21, 2025

(51) Int. Cl. A61N 5/10; A61B 17/00

(52) U.S. Cl. CPC A61N 5/1071; A61B 2017/00907; A61N 2005/1021; A61N 2005/1094

(57) ABSTRACT

A radiation delivery determination system includes a container that includes a first compartment defining an internal cut-out sized and shaped to receive a periphery of a radioembolization administration set and a second compartment defining a pocket configured to receive tubing, where the tubing is in fluid communication with and attached to the radioembolization administration set. The radiation delivery determination system further includes a radioactive measurement device external to the container and configured to measure an amount of radiation within the radioembolization administration set and the tubing.

### TECHNICAL FIELD

The present disclosure generally relates to components of medical devices for treating diseases such as cancer, and more particularly to systems and methods for determining an amount of radiation within at least a portion of a medical treatment device.

### BACKGROUND

In treatments involving radiation therapy, such as cancer treatments, misadministration of a radioactive dose may compromise the efficacy of a treatment procedure. Therefore, in order to ensure that a predetermined amount of radiation has been administered to a patient, an amount of radiation still remaining in a medical treatment device may be measured following a treatment operation. However, due to the specific geometries of various components of the medical treatment device and different materials used for different components of the medical treatment device, it may be difficult to obtain accurate and repeatable measurements of the amount of radiation remaining in the medical treatment device following a treatment procedure.

Accordingly, a need exists for systems and methods for reproducibly and accurately determining an amount of radiation within at least a portion of a medical treatment device.

### SUMMARY

In accordance with an embodiment of the disclosure, a radiation delivery determination system may include a container. The container includes a first compartment defining an internal cut-out sized and shaped to receive a periphery of a radioembolization administration set and a second compartment defining a pocket configured to receive tubing, where the tubing is in fluid communication with and attached to the radioembolization administration set. The radiation delivery determination system further includes a radioactive measurement device external to the container and configured to measure an amount of radiation within the radioembolization administration set and the tubing.

In another embodiment, a radiation delivery determination system may include a container that includes a first compartment defining an internal cut-out sized and shaped to receive a periphery of a radioembolization administration set, where the radioembolization administration set includes an at least partially administered vial of radioactive therapeutic material, and a second compartment defining a pocket configured to receive tubing, where the tubing is in fluid communication with and attached to the radioembolization administration set. The tubing is disposed within the pocket of the second compartment of the container, and the tubing includes at least a first tube for administering radioactive therapeutic material from the vial. The radiation delivery determination system also includes a Geiger counter external to the container and configured to measure a total amount of radiation within the radioembolization administration set and the tubing.

In yet another embodiment, a method of measuring an amount of radiation within a radioembolization administration set and tubing in fluid communication with the radioembolization administration set may include placing the radioembolization administration set and the tubing in a container. The container includes a first compartment defining an internal cut-out sized and shaped to receive a periphery of the radioembolization administration set, where the radioembolization administration set is placed within the internal cut-out of the first compartment, and a second compartment defining a pocket configured to receive the tubing, where the tubing is placed within the pocket of the second compartment. The method also includes measuring the amount of radiation within the radioembolization administration set and the tubing with a radioactive measurement device disposed external to and spaced from the container.

These and additional features provided by the embodiments described herein will be more fully understood in view of the following detailed description, in conjunction with the drawings.

### BRIEF DESCRIPTION OF THE DRAWINGS

FIG. 1 is a perspective view of a delivery device including a protective shield and a radioembolization administration set, according to one or more embodiments shown and described herein;

FIG. 2 is a cross-sectional view of the radioembolization administration set of FIG. 1, according to one or more embodiments shown and described herein, the cross-section along line 2 -2 of FIG. 1;

FIG. 3 is a perspective view of a vial assembly including an engagement head, according to one or more embodiments shown and described herein;

FIG. 4 is a perspective view of the radioembolization administration set of FIG. 1 with the vial assembly of FIG. 3 received therein, with a series of delivery lines coupled to the radioembolization administration set, according to one or more embodiments shown and described herein;

FIG. 5 is a perspective view of a container for receiving the radioembolization administration set of FIG. 4 therein, the front wall of the container removed for illustration, according to one or more embodiments shown and described herein;

FIG. 6 is a perspective view of the container of FIG. 5 with the radioembolization administration set of FIG. 4 received therein, according to one or more embodiments shown and described herein;

FIG. 7 is a perspective view of a liner of the container of FIG. 5 for receiving the radioembolization administration set of FIG. 4 therein, according to one or more embodiments shown and described herein;

FIG. 8 is a top view of the container of FIG. 5 with a top wall removed for illustration and including a front wall forming a projection, according to one or more embodiments shown and described herein;

FIG. 9 is a top view of another embodiment of the container of FIG. 5 with a top wall removed for illustration and including a front wall forming a contour, according to one or more embodiments shown and described herein;

FIG. 10 is a perspective view of another container for receiving the radioembolization administration set of FIG. 4 therein, the front wall of the container removed for illustration, according to one or more embodiments shown and described herein;

FIG. 11 is a cross-sectional view of the container of FIG. 10, according to one or more embodiments shown and described herein, the cross-section along line 10 -10 of FIG. 10, according to one or more embodiments shown and described herein;

FIG. 12 is a cross-sectional view of the container of FIG. 10, according to one or more embodiments shown and described herein, the cross-section along line 11 -11 of FIG. 10, according to one or more embodiments shown and described herein;

FIG. 13 is a perspective view of a casing for receiving a container for receiving the radioembolization administration set of FIG. 4 therein, according to one or more embodiments shown and described herein;

FIG. 14 is a perspective view of the casing of FIG. 13 with the container of FIG. 6 received therein, according to one or more embodiments shown and described herein;

FIG. 15 is a flowchart for determining an amount of radiation within at least a portion of a medical treatment device, according to one or more embodiments shown and described herein.

### DETAILED DESCRIPTION

Reference will now be made in detail to various embodiments of delivery devices for administering radioactive compounds to a patient, examples of which are illustrated in the accompanying drawings. Whenever possible, the same reference numerals will be used throughout the drawings to refer to the same or like parts. Directional terms as used herein-for example up, down, right, left, front, back, top, bottom, distal, and proximal-are made only with reference to the figures as drawn and are not intended to imply absolute orientation.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

Unless otherwise defined, all technical and scientific terms used herein have the same meaning as commonly understood by one of ordinary skill in the art to which the disclosure belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

As used herein, the terms “horizontal,” “vertical,” “distal” and “proximal” are relative terms only, are indicative of a general relative orientation only, and do not necessarily indicate perpendicularity. These terms also may be used for convenience to refer to orientations used in the figures, which orientations are used as a matter of convention only and are not intended as characteristic of the devices shown. The present disclosure and the embodiments thereof to be described herein may be used in any desired orientation. Moreover, horizontal and vertical walls need generally only be intersecting walls, and need not be perpendicular. As used herein, the singular forms “a,” “an” and “the” include plural referents unless the context clearly dictates otherwise. Thus, for example, reference to “a” component includes aspects having two or more such components, unless the context clearly indicates otherwise.

In embodiments described herein, a particulate material delivery assembly for which an amount of radiation may be determined as described in greater detail further below may include a radioembolization administration set. A radioembolization administration set comprises a medical device such as a medical treatment device configured to deliver radioactive compounds to a treatment area within a patient's body in procedures such as transarterial radioembolization. The radioactive compounds may be a mixed solution of saline and radioactive microspheres (i.e., a particulate) mixed in a vial of a vial assembly. The needle may include one or more ports as an outlet to inject fluid (i.e., saline), such as from a syringe or catheter line, into a vial including the radioactive microspheres to generate the mixed solution and as an inlet to deliver the mixed solution to the patient.

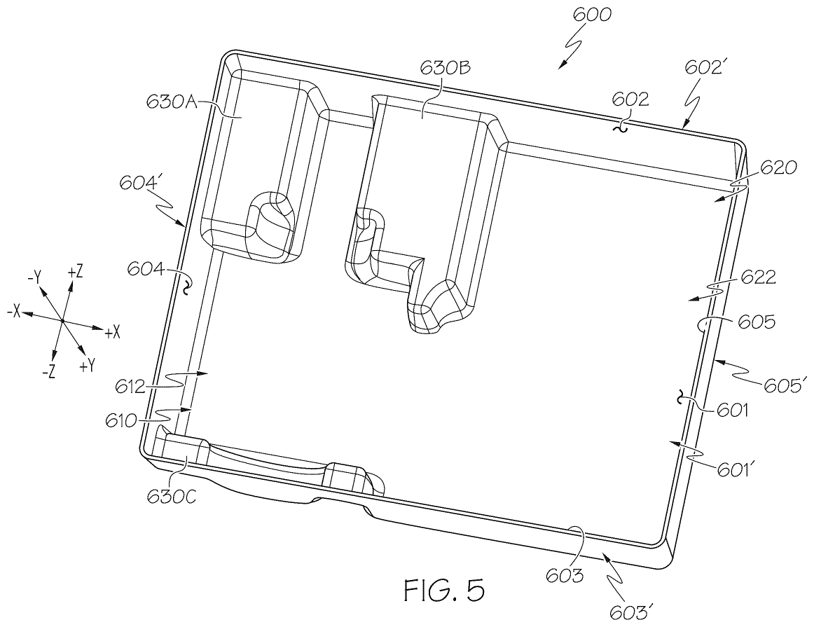

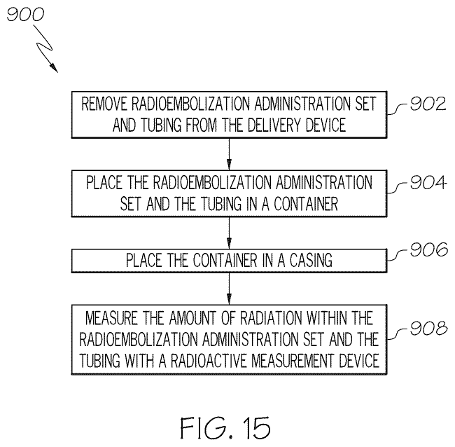

Also in embodiments described herein and described in greater detail further below with respect to at least FIGS. 5-15, a radiation delivery determination system for determining an amount of radiation within the radioembolization administration set, which may include the vial assembly received therein, and tubing coupled to the radioembolization administration set includes a container. The container includes a first compartment defining an internal cut-out sized and shaped to receive a periphery of a radioembolization administration set and a second compartment defining a pocket configured to receive tubing. In embodiments, to normalize a reduction of radiation emission intensity of radiation contained within the radioembolization administration set and/or the vial assembly and the tubing, a rear first surface of the first compartment disposed opposite a measurement-facing surface of a front wall of the container is spaced a first distance from the measurement-facing surface of the container, and a rear second surface of the second compartment disposed opposite the measurement-facing surface of the container is spaced a second distance from the measurement-facing surface of the container, where the second distance is greater than the first distance. The measurement-facing surface of the front wall of the container is nearest to and facing the radioactive measurement device. In additional or alternative embodiments, to normalize a reduction of radiation emission intensity of radiation contained within the radioembolization administration set and/or the vial assembly and the tubing, the container is placed within a casing that includes a radioactive shield that partially extends along the casing and covers the second compartment of the container, but does not cover the first compartment of the container.

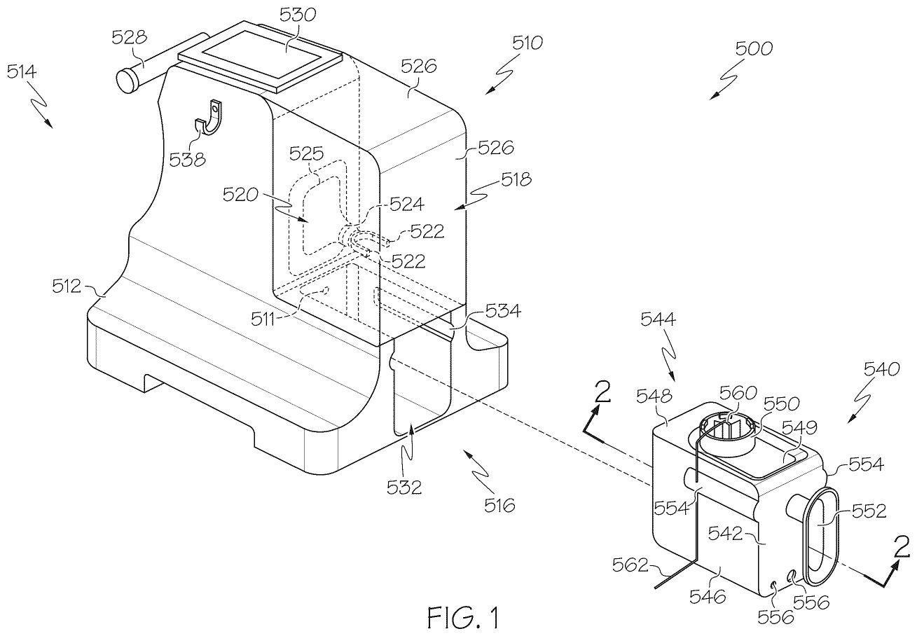

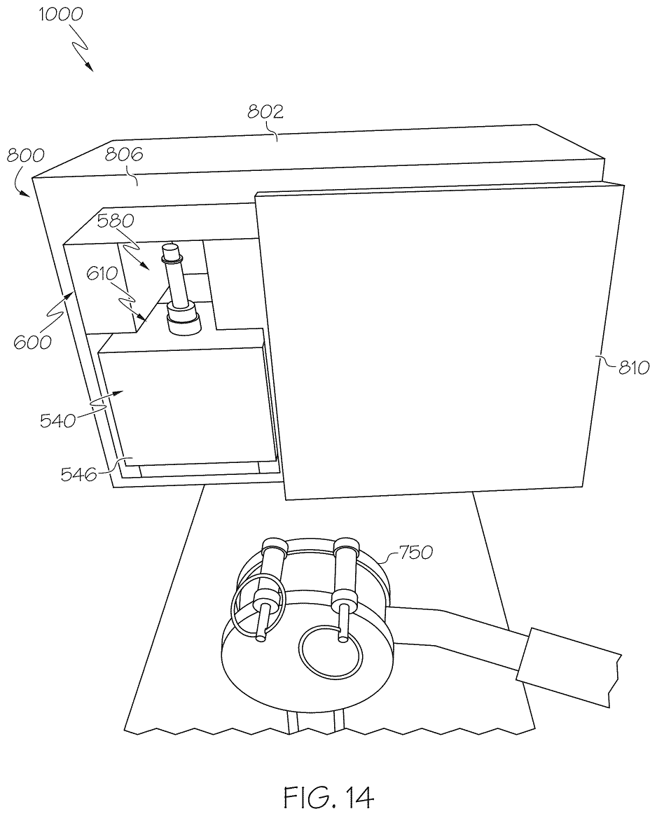

FIGS. 1-4 described below are directed to an embodiment of a delivery device 500 to deliver a particulate, and FIGS. 5-15 described in greater detail further below are directed to embodiments of one or more systems 1000 (see FIG. 14) to assist with determining an amount of radiation within at least a portion of the delivery device 500. In some embodiments, as described in greater detail below, the delivery device 500 is a radioembolization delivery device, the particulate is a plurality of radioembolization beads, the fluid is a saline solution, and the resulting mixed fluid (e.g., the mixed fluid solution) is a radioembolization beads-saline solution. The needle 559 may be configured to deliver the radioembolization beads-saline solution as the mixed fluid solution through the radioembolization delivery device, such as upon actuation of the vial engagement mechanism 520 in the positive pressure direction. In some embodiments, the fluid is a contrast-saline solution including a contrast agent, and the resulting mixed fluid (e.g., the mixed fluid solution) is a radioembolization beads-contrast-saline solution. The needle 559 may be configured to deliver the radioembolization beads-contrast-saline solution as the mixed fluid solution through the radioembolization delivery device. In some embodiments, the delivery device 500 is a chemoembolization delivery device, the particulate is a plurality of chemoembolization beads, and the mixed fluid solution is a beads-saline solution or a beads-contrast-saline solution.

### I. Mechanical Delivery Device with Removable Radioembolization Administration Set

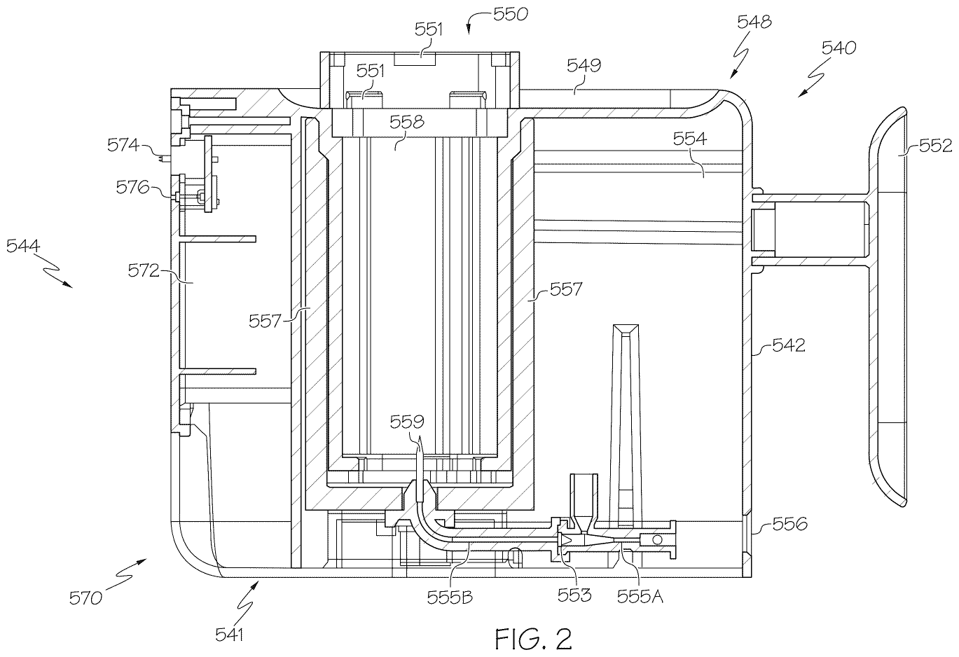

FIGS. 1 and 2 show an embodiment of a delivery device 500 that is configured and operable to deliver a radioactive material (e.g., radioembolizing beads) while reducing radioactive emissions during use of the delivery device 500. The delivery device 500 may operate as described in International PCT App. No. PCT/2019/033001, filed May 17, 2019, the entirety of which is incorporated herein.

Referring initially to FIG. 1, the delivery device 500 comprises a console assembly 510, which includes a console. The delivery device 500 may include a radioembolization administration set 540 that is operable to transition between a coupled state and decoupled state relative to the console assembly 510. The console assembly 510 of the delivery device 500 comprises a base 512 defined by and extending between a proximal end 514 and a distal end 516. The proximal end 514 of the base 512 includes a handle (delivery handle) 528 movably coupled to the console assembly 510 and an interface display 530 positioned on the console assembly 510.

The proximal end 514 of the base 512 further includes an attachment device 538 that is configured to securely retain an external device to the base 512 of the console assembly 510. The attachment device 538 is operable to facilitate an attachment of a complimentary device to the console assembly 510 for use with the delivery device 500 during a procedure.

Still referring to FIG. 1, the distal end 516 of the console assembly 510 defines a vial containment region 518 that is sized and shaped to receive the console assembly 510 therein, as will be described in greater detail herein. The console assembly 510 further includes a vial engagement mechanism 520 extending from the base 512 adjacent to the distal end 516. In particular, the vial engagement mechanism 520 extends laterally outward from the base 512 of the console assembly 510 toward the distal end 516. The vial engagement mechanism 520 is positioned within the vial containment region 518 of the console assembly 510 and is movably coupled to the handle 528. In particular, the handle 528 of the console assembly 510 is operable to move, and in particular translate, the vial engagement mechanism 520 within the vial containment region 518 in response to an actuation of the handle 528.

The console assembly 510 includes a mechanical assembly disposed within the base 512 that is configured and operable to convert a manual motion of the handle 528 to a corresponding linear displacement of the vial engagement mechanism 520. In the present example, the mechanical assembly is coupled to the handle 528 and the vial engagement mechanism 520 such that selective actuation of the handle 528 at the proximal end 514 causes a simultaneous actuation of the vial engagement mechanism 520 at the distal end 516.

In embodiments, and referring to FIG. 2, a flow sensor of the delivery device 500 may be positioned in-line with the tubing set of the delivery device 500, and in particular the needle 559, the manifolds 555A, 555B, and/or one or more of the ports 556, and may be configured to measure an amount of fluid (e.g., suspension liquid after the therapeutic particles have effectively mixed with the fluid medium) that passes thereby. Referring back to FIG. 1, the vial engagement mechanism 520 comprises a pair of lever arms 522 extending outwardly from a neck 524 of the vial engagement mechanism 520, with the neck 524 extending laterally outward from the base 512 of the console assembly 510. The neck 524 of the vial engagement mechanism 520 is disposed within a protective cover 525 such that only the pair of lever arms 522 of the vial engagement mechanism 520 extends through the protective cover 525. The protective cover 525 is operable to shield one or more internal components of the console assembly 510 from an exterior of the console assembly 510, and in particular from the vial containment region 518.

The pair of lever arms 522 is simultaneously movable with the neck 524 of the vial engagement mechanism 520 in response to an actuation of the handle 528 of the console assembly 510. Further, the pair of lever arms 522 are fixed relative to one another such that a spacing formed between the pair of lever arms 522 is relatively fixed. The pair of lever arms 522 of the vial engagement mechanism 520 is configured to securely engage the vial assembly 580 therebetween, and in particular within the spacing formed by the pair of lever arms 522. Accordingly, the vial engagement mechanism 520 is operable to securely attach the vial assembly 580 to the console assembly 510 at the vial containment region 518. Although the vial engagement mechanism 520 is shown and described herein as including a pair of lever arms 522, it should be understood that the vial engagement mechanism 520 may include various other structural configurations suitable for engaging the vial assembly 580, such as magnets on each component configured to engage with one another.

Still referring to FIG. 1, the console assembly 510 further includes a safety shield 526 secured to the distal end 516 of the base 512 along the vial containment region 518. In particular, the safety shield 526 is a protective covering that is sized and shaped to enclose the vial containment region 518 of the console assembly 510 when secured thereon. The safety shield 526 is selectively attachable to the distal end 516 of the base 512 and is formed of a material that is configured to inhibit radioactive emissions from one or more radioactive doses stored within the vial containment region 518, such as a glass, polymer, or other plastic material.

The distal end 516 of the console assembly 510 further includes an administration set cavity 532 that is sized and shaped to receive the radioembolization administration set 540 therein. The administration set cavity 532 includes one or more, and in some embodiments, a pair of alignment features 534 extending therein, with the alignment features 534 sized and shaped to correspond with complimentary alignment features of the radioembolization administration set 540 (e.g., alignment ribs 554) to thereby facilitate a coupling of the radioembolization administration set 540 with the base 512 of the console assembly 510 within the administration set cavity 532. As will be described in greater detail herein, the radioembolization administration set 540 is configured to store and administer therapeutic particles (e.g., radioactive beads, microspheres, medium) therethrough. In particular, the radioembolization administration set 540 is configured to partially receive a vial assembly 580 therein for administering the therapeutic particles from the delivery device 500 and to a patient during a procedure.

Still referring to FIG. 1, the radioembolization administration set 540 is configured to partially receive a vial assembly 580 therein for administering therapeutic particles (e.g., radioactive fluid medium) from the delivery device 500 and to a patient. In particular, the radioembolization administration set 540 comprises a proximal end 544 and a distal end 542 with a pair of sidewalls 546 extending therebetween. The proximal end 544 of the radioembolization administration set 540 includes a handle 552 extending proximally therefrom. The handle 552 is configured to facilitate movement of the radioembolization administration set 540, and in particular, an insertion of the radioembolization administration set 540 into the administration set cavity 532 of the console assembly 510. The proximal end 544 further includes one or more ports 556 for coupling one or more delivery lines (i.e., tubing) to the radioembolization administration set 540. With the one or more delivery lines further be coupled to one or more external devices at an end of the line opposite of the ports 556, the ports 556 effectively serve to fluidly couple the radioembolization administration set 540 to the one or more external devices via the delivery lines connected thereto. The pair of sidewalls 546 of the radioembolization administration set 540 includes at least one alignment rib 554 extending laterally outward therefrom, where the alignment ribs 554 are sized and shaped to correspond with and mate to the pair of alignment features 534 of the console assembly 510. Accordingly, the pair of alignment ribs 554 are configured to facilitate an alignment and engagement of the radioembolization administration set 540 with the console assembly 510 when the distal end 542 is slidably received within the administration set cavity 532 of the base 512.

The radioembolization administration set 540 further includes a top surface 548 extending from the proximal end 544 and the distal end 542 and positioned between the pair of sidewalls 546, and a bottom surface 541 extending from the proximal end 544 and the distal end 542 and positioned between the pair of sidewalls 546. The top surface 548 of the radioembolization administration set 540 includes a recessed region 549 and a locking system 550. The recessed region 549 is sized and shaped to form a recess and/or cavity along the top surface 548, where the recessed region 549 is capable of receiving and/or collecting various materials therein, including, for example, leaks of various fluid media during use of the delivery device 500. The locking system 550 of the radioembolization administration set 540 forms an opening along the top surface 548 that is sized and shaped to receive one or more devices therein, such as a priming assembly 560 and a vial assembly 580. In some embodiments, the radioembolization administration set 540 comes preloaded with the priming assembly 560 disposed within the locking system 550. The priming assembly 560 includes a priming line 562 extending outwardly from the locking system 550 of the radioembolization administration set 540. The priming assembly 560 connects the priming line 562 to needle 559 and manifolds 555A and 555B and serves to purge the delivery device 500, including the manifolds 555A and 555B, of air prior to utilizing the delivery device 500 in a procedure.

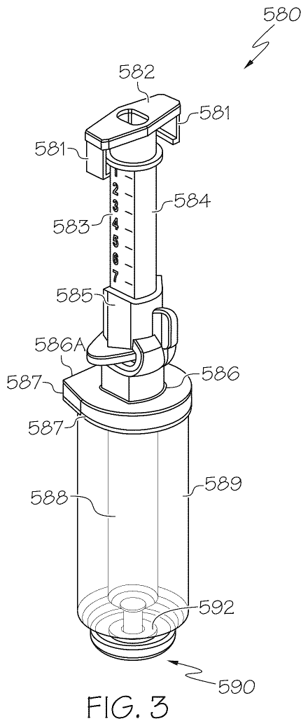

Referring now to FIG. 2, the locking system 550 includes an annular array of one or more projections 551 extending outwardly therefrom, and in particular, extending laterally into the aperture formed by the locking system 550 along the top surface 548. The annular array of projections 551 are formed within an inner perimeter of the locking system 550 and extend along at least two sequentially-arranged rows. The annular array of projections 551 included in the locking system 550 are configured to engage a corresponding locking feature 586 of the vial assembly 580 (See FIG. 3) to thereby securely fasten the vial assembly 580 to the radioembolization administration set 540. It should be understood that the multiple rows of projections 551 of the locking system 550 serve to provide a double-locking system to ensure the radioembolization administration set 540, and in particular a needle 559 of the radioembolization administration set 540, is securely maintained through a septum 592 of the vial assembly 580 (See FIG. 3) during use of the delivery device 500 in a procedure.

The radioembolization administration set 540 further includes a vial chamber 558 that is sized and shaped to receive the priming assembly 560 and the vial assembly 580 therein, respectively. In other words, the vial chamber 558 is sized to individually receive both the priming assembly 560 and the vial assembly 580 separate from one another. The vial chamber 558 is encapsulated around a protective chamber or shield 557 disposed about the vial chamber 558. The protective shield 557 is formed of a material configured to inhibit radioactive emissions from extending outwardly from the vial chamber 558, such as, for example, a metal. Additionally, the radioembolization administration set 540 includes a needle 559 extending through the protective shield 557 and into the vial chamber 558 along a bottom end of the vial chamber 558. The needle 559 is fixedly secured relative to the vial chamber 558 such that any devices received through the aperture of the locking system 550 and into the vial chamber 558 are to encounter and interact with the needle 559 (e.g., the priming assembly 560, the vial assembly 580, and the like).

Still referring to FIG. 2, the needle 559 is coupled to a distal manifold 555A and a proximal manifold 555B disposed within the radioembolization administration set 540, and in particular the manifold 555A, 555B is positioned beneath the vial chamber 558 and the protective shield 557. The proximal manifold 555B is fluidly coupled to the needle 559 and the distal manifold 555A is fluidly couplable to one or more delivery lines via the one or more ports 556 of the radioembolization administration set 540. The proximal manifold 555B is in fluid communication with the distal manifold 555A through a one-way check valve 553 disposed therebetween.

Accordingly, the proximal manifold 555B is in fluid communication with the one or more ports 556 via the distal manifold 555A, however, the one or more ports 556 are not in fluid communication with the proximal manifold 555B due to a position of the one-way check valve 553 disposed between the manifolds 555A, 555B. Thus, the needle 559 is in fluid communication with the one or more delivery lines and/or devices coupled to the radioembolization administration set 540 at the one or more ports 556 via the manifolds 555A, 555B secured therebetween. The one or more ports 556 of the radioembolization administration set 540 may be coupled to a bag (e.g., saline bag), a syringe, a catheter, and/or the like via one or more delivery lines coupled thereto. In other embodiments, the needle 559 may be a cannula, catheter, or similar mechanism through which to inject and receive fluid and/or a solution as described herein.

Still referring to FIG. 2, the radioembolization administration set 540 includes a removable battery pack 570 coupled to the radioembolization administration set 540 along the distal end 542. The removable battery pack 570 comprises a battery 572, electrical contacts 574, and a removable tab 576. The battery 572 of the delivery device 500 is isolated from one or more fluid paths and radiation sources due to a location of the battery 572 in the removable battery pack 570.

The electrical contacts 574 of the removable battery pack 570 extend outwardly from the removable battery pack 570 and are operable to contact against and interact with corresponding electrical contacts 511 of the console assembly 510 (See FIG. 1) when the radioembolization administration set 540 is coupled to the base 512 at the administration set cavity 532. Accordingly, the removable battery pack 570 is operable to provide electrical power to the delivery device 500, and in particular the console assembly 510, when the radioembolization administration set 540 is coupled to the console assembly 510.

Additionally, as will be described in greater detail herein, in some embodiments the locking system 550 may include at least one planar wall relative to a remaining circular orientation of the locking system 550. In this instance, an aperture formed by the locking system 550 through the top surface 548 of the radioembolization administration set 540 is irregularly-shaped, rather than circularly-shaped as shown and described above. In this instance, the vial assembly 580 includes a locking feature 586 that has a shape and size that corresponds to the locking system 550, and in particular the at least one planar wall such that the vial assembly 580 is received within the radioembolization administration set 540 only when an orientation of the vial assembly 580 corresponds with an alignment of the locking feature 586 and the locking system 550. In other words, a corresponding planar wall 586A of the locking feature 586 (See FIG. 3) must be aligned with the planar wall of the locking system 550 for the vial assembly 580 to be receivable within an aperture formed by the locking system 550 of the radioembolization administration set 540.

Referring now to FIG. 3, the vial assembly 580 of the delivery device 500 is depicted. The vial assembly 580 comprises an engagement head 582, a plunger 584, a locking feature 586, and a vial body 589. In particular, the engagement head 582 of the vial assembly 580 is positioned at a terminal end of the plunger 584 opposite of the locking feature 586 and the vial body 589. The engagement head 582 includes a pair of arms 581 extending laterally outward relative to a longitudinal length of the plunger 584 extending downwardly therefrom. In the present example, the engagement head 582 is integrally formed with the plunger 584, however, it should be understood that in other embodiments the engagement head 582 and the plunger 584 may be separate features fastened thereto. In either instance, the engagement head 582 and the plunger 584 is movable relative to the locking feature 586 and the vial body 589 such that the engagement head 582 and the plunger 584 are slidably translatable through the locking feature 586 and the vial body 589. In particular, as will be described in greater detail herein, the plunger 584 may translate into and out of an internal chamber 588 of the vial body 589 in response to a linear translation of the vial engagement mechanism 520 when the engagement head 582 is secured to the pair of lever arms 522.

The plunger 584 includes a plurality of indicia and/or markings 583 positioned along a longitudinal length of the plunger 584. The plurality of markings 583 is indicative of a relative extension of the engagement head 582 and the plunger 584 from the locking feature 586 and the vial body 589. As briefly noted above, the engagement head 582 is configured to attach the vial assembly 580 to the vial engagement mechanism 520. In particular, the pair of arms 581 of the engagement head 582 are sized and shaped to couple with the pair of lever arms 522 of the vial engagement mechanism 520 when the vial assembly 580 is received within the radioembolization administration set 540 and the radioembolization administration set 540 is inserted into the administration set cavity 532 of the console assembly 510. As will be described in greater detail herein, the pair of lever arms 522 are received between the pair of arms 581 of the engagement head 582 and the plunger 584 in response to a predetermined translation force applied to the vial engagement mechanism 520. The engagement head 582 and the plunger 584 may be formed of various materials, including, but not limited to, a metal, plastic, and/or the like.

Still referring to FIG. 3, the vial assembly 580 further includes a safety tab 585 coupled to the plunger 584 relatively above the locking feature 586 and below the engagement head 582 such that the safety tab 585 is positioned along the longitudinal length of the plunger 584. The safety tab 585 may be formed of various materials, such as, for example, a plastic, and is preassembled onto the vial assembly 580 prior to a use of the delivery device 500. The safety tab 585 is removably fastened to the plunger 584 and inhibits the plunger 584 from translating relative to the vial body 589. In particular, the safety tab 585 abuts against the locking feature 586 in response to an application of linear force onto the plunger 584 to translate the plunger 584 relatively downward into the vial body 589. In this instance, the safety tab 585 is configured to inhibit an inadvertent movement of the plunger 584, and in response, an inadvertent delivery of a fluid media stored within the internal chamber 588 of the vial body 589 (e.g., therapeutic particles, radioembolizing beads). As will be described in greater detail herein, the safety tab 585 is selectively disengaged from the plunger 584 in response to a coupling of the vial assembly 580 with the vial engagement mechanism 520, and in particular an engagement of the pair of lever arms 522 with the engagement head 582.

Referring back to FIG. 3, the locking feature 586 extends about a top end of the vial body 589. In the present example, the locking feature 586 of the vial assembly 580 comprises a bushing that defines a lateral edge 587 extending laterally outward along an outer perimeter of the locking feature 586. The lateral edge 587 of the locking feature 586 is sized and shaped to engage the annular array of projections 551 of the locking system 550 when the vial assembly 580 is received within the vial chamber 558 of the radioembolization administration set 540. As will be described in greater detail herein, the locking feature 586, and in particular the lateral edge 587 of the locking feature 586, is configured to securely fasten the vial assembly 580 to the locking system 550 to inhibit removal of the vial body 589 from the vial chamber 558 of the radioembolization administration set 540 during use of the delivery device 500 in a procedure. In some embodiments, as briefly described above, the locking feature 586 includes at least one planar wall 586A such that the locking feature 586 comprises an irregular-profile. The at least one planar wall 586A is configured to correspond to the planar wall 550A of the locking system 550 such that an alignment of the planar walls 550A, 586A is required for the vial assembly 580 to be received through an aperture formed by the locking system 550.

Still referring to FIG. 3, the vial body 589 extends downwardly relative from the locking feature 586 and has a longitudinal length that is sized to receive at least a portion of a longitudinal length of the plunger 584 therein. By way of example only, a longitudinal length of the vial body 589 may be about 8 millimeters to about 10 millimeters, and in the present example comprises 9 millimeters, while a longitudinal length of the plunger 584 may be about 9 millimeters to about 11 millimeters, and in the present example comprises 10 millimeters. Accordingly, in some embodiments a longitudinal length of the plunger 584 exceed a longitudinal length of the vial body 589 such that a translation of the plunger 584 into the internal chamber 588 of the vial body 589 causes a fluid media stored therein to be transferred outward from the vial body 589. As will be described in greater detail herein, a translation of the plunger 584 through the internal chamber 588 of the vial body 589 provides for an administration of a fluid media stored within the vial body 589 outward from the vial assembly 580. The vial body 589 may be formed of various materials, including, for example, a thermoplastic polymer, copolyester, polycarbonate, a biocompatible plastic, polysulfone, ceramics, metals, and/or the like.

The vial body 589 is of the present example is formed of a material that is configured to inhibit radioactive emissions from a fluid media stored within the internal chamber 588 of the vial body 589. For example, the vial body 589 may be formed of a plastic, such as polycarbonate, and have a width of approximately 9 millimeters (mm). A density and material composition of the vial body 589 may collectively inhibit beta radiation emission from electron particles stored within the internal chamber 588. In the present example, a chemical composition of the plastic of the vial body 589, along with the 9 mm wall thickness, provides a plurality of atoms disposed within the vial body 589 that are capable of encountering the electron particles generating beta radiation and reducing an emission of said radiation from the vial assembly 580. Accordingly, the vial assembly 580 allows an operator to handle the radioactive material stored within the vial body 589 without being exposed to beta radiation. It should be understood that various other materials and/or wall sections may be incorporated in the vial body 589 of the vial assembly 580 in other embodiments without departing from the scope of the present disclosure.

Still referring to FIG. 3, the vial body 589 of the vial assembly 580 is sealed at a first terminal end by the locking feature 586. The vial assembly 580 further includes a cap 590 positioned at an opposing, terminal end of the vial body 589 opposite of the locking feature 586, such that the cap 590 seals a second terminal end of the vial body 589 of the vial assembly 580. Additionally, the vial assembly 580 includes a septum 592 positioned adjacent to the cap 590 and in fluid communication with a terminal end of the vial body 589 opposite of the locking feature 586. The septum 592 forms a seal against a terminal end of the vial body 589 and the cap 590 retains the septum 592 therein. The septum 592 may be formed of various materials, including, for example, an elastomer, silicon, bromobutyl elastomer, rubber, urethanes, and/or the like. The septum 592 is configured to provide an air-tight seal for the vial body 589 to thereby inhibit a release of a fluid media stored therein (e.g., radioembolizing beads). As will be described in greater detail herein, the septum 592 of the vial assembly 580 is configured to be punctured by the needle 559 of the radioembolization administration set 540 when the vial assembly 580 is received within the vial chamber 558, thereby establishing fluid communication between the vial body 589 and the radioembolization administration set 540. In other embodiments, the septum 592 may be omitted entirely for an alternative device, such as, for example, a valve system, needle injection port, and/or the like.

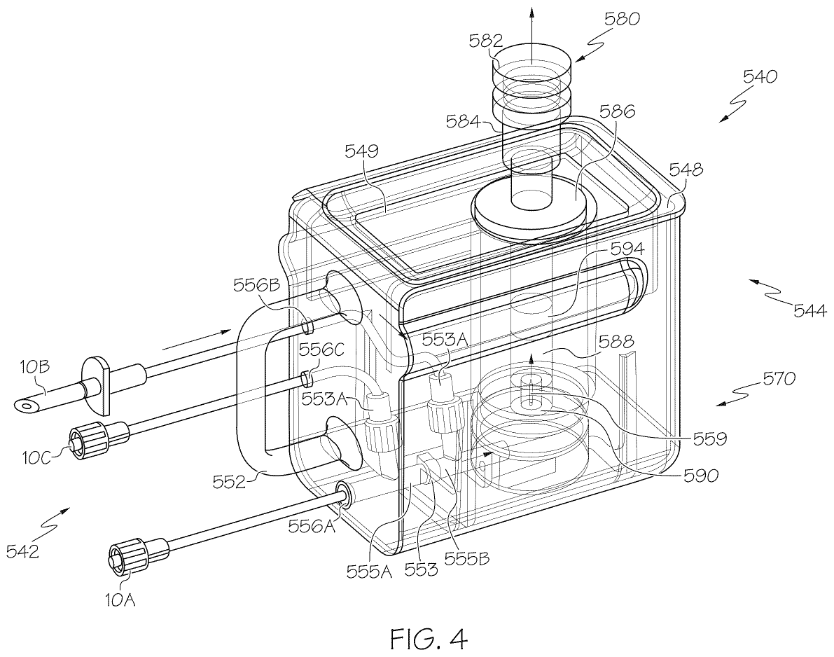

Referring now to FIG. 4, in response to determining that the battery 572 contains or other power source provides a sufficient amount of power, one or more delivery lines are coupled to the radioembolization administration set 540 via the one or more ports 556. In particular, a dose delivery line 10A is coupled to the radioembolization administration set 540 at a delivery port 556A, a contrast line 10B is coupled to radioembolization administration set 540 at a contrast port 556B, and a flushing line 10C is coupled to the radioembolization administration set 540 at a flushing port 556C. An opposing end of the dose delivery line 10A is initially coupled to a fluid reservoir, such as, for example, a collection bowl. As will be described in greater detail herein, the dose delivery line 10A may be subsequently coupled to an external device, such as a catheter, once the radioembolization administration set 540 has been effectively primed by a fluid medium via the contrast line 10B. An opposing end of the flushing line 10C is coupled to an external device, such as, for example, a syringe. With both the dose delivery line 10A and the flushing line 10C coupled to the radioembolization administration set 540, the radioembolization administration set 540 is flushed with a fluid medium (e.g., saline) from the syringe coupled to the flushing line 10C. In this instance, the fluid medium is injected through the flushing line 10C, into the distal manifold 555A of the radioembolization administration set 540, and out of the radioembolization administration set 540 through the dose delivery line 10A. Accordingly, the fluid medium is ultimately received at the collection bowl and disposed thereat by the dose delivery line 10A.

With the distal manifold 555A of the radioembolization administration set 540 separated from the proximal manifold 555B by the one-way check valve 553 disposed therebetween, the fluid medium flushed through the distal manifold 555A from the syringe (via the flushing port 556C) is prevented from passing through the proximal manifold 555B and the needle 559 coupled thereto. Rather, the fluid medium injected from the syringe and through the flushing line 10C is received at the flushing port 556C, passed through the distal manifold 555A in fluid communication with the flushing port 556C, and redirected by the one-way check valve 553 towards the dose delivery port 556A that is coupled to the dose delivery line 10A. In this instance, the dose delivery line 10A receives and transfers the fluid medium to the collection bowl coupled thereto, such that the fluid medium is not directed beyond the one-way check valve 553 and into the proximal manifold 555B that is in fluid communication with the needle 559.

The contrast line 10B is coupled to the radioembolization administration set 540 at a contrast port 556B. An opposing end of the contrast line 10B is coupled to a fluid medium supply, such as, for example, a bag secured to the console assembly 510 via the attachment device 538. In the present example, the bag is a saline bag such that the fluid medium stored therein is saline. In this instance, with the radioembolization administration set 540 including the priming assembly 560 positioned within the vial chamber 558 and a needle end in fluid communication with the needle 559, a syringe is fluidly coupled to the priming line 562 of the priming assembly 560 and a plunger of the syringe is drawn back to pull saline through the contrast line 10B, the contrast port 556B, the radioembolization administration set 540, the priming line 562 and into the syringe from the saline bag. The plunger of the syringe is thereafter pushed inwards to transfer the extracted saline back through the priming line 562, a central body, an elongated shaft, and the needle end of the priming assembly 560 such that the saline is received into the needle 559 of the radioembolization administration set 540. Accordingly, the manifolds 555A, 555B of the radioembolization administration set 540 are effectively primed with the saline from the syringe as the needle 559 that received the saline from the priming assembly 560 is in fluid communication with the manifolds 555A, 555B. With the manifolds 555A, 555B in further fluid communication with the dose delivery line 10A via the delivery port 556A, the saline is effectively distributed to the collection bowl coupled thereto.

Referring now to FIG. 4, the radioembolization administration set 540 is coupled to one or more external devices via the one or more ports 556. In particular, the radioembolization administration set 540 is fluidly coupled to a catheter (e.g., microcatheter) via the dose delivery line 10A that is coupled to the delivery port 556A of the radioembolization administration set 540. In this instance, the catheter is in fluid communication with the radioembolization administration set 540 via the dose delivery line 10A. Further, the radioembolization administration set 540 may be fluidly coupled to a contrast source, such as, for example, a saline bag secured to the console assembly 510 via the attachment device 538 (See FIG. 1). The radioembolization administration set 540 is in fluid communication with the saline bag via a contrast line 10B coupled to the contrast port 556B of the radioembolization administration set 540. In this instance, the saline bag is in fluid communication with the radioembolization administration set 540 via the contrast line 10B secured to the contrast port 556B.

The contrast port 556B is in fluid communication with the proximal manifold 555B while the delivery port 556A is in fluid communication with the distal manifold 555A. As will be described in greater detail herein, saline from the saline bag may be withdrawn through the needle 559 of the radioembolization administration set 540 and into the vial body 589 of the vial assembly 580 as the contrast port 556B is coupled to the proximal manifold 555B, rather than the distal manifold 555A which is separated from the proximal manifold 555B by the one-way check valve 553 disposed therebetween.

Referring again to FIGS. 1 and 3, with the vial assembly 580 securely coupled to the radioembolization administration set 540, the radioembolization administration set 540 is coupled to the console assembly 510 by translating the proximal end 544 of the radioembolization administration set 540 toward and into the distal end 516 of the console assembly 510. In particular, the proximal end 544 of the radioembolization administration set 540 is directed into the administration set cavity 532 of the console assembly 510 by aligning the alignment ribs 554 of the radioembolization administration set 540 with the alignment features 534 of the console assembly 510. Once the distal end 542 and the proximal end 544 of the radioembolization administration set 540 are fully seated within the administration set cavity 532 of the console assembly 510, the electrical contacts 574 (FIG. 2) of the removable battery pack 570 interact with corresponding electrical contacts 511 (FIG. 1) of the console assembly 510. In this instance, power from the battery 572 is transmitted to the console assembly 510 via the electrical contacts 574, thereby activating the console assembly 510 of the delivery device 500. In this instance, the interface display 530 of the console assembly 510 is activated to display pertinent, real-time information relating to the delivery device 500 during a procedure.

Referring again to FIG. 4, as the vial engagement mechanism 520 and the plunger 584 are simultaneously translated within the vial containment region 518, a negative pressure is generated within the internal chamber 588 of the vial body 589 due to a retraction of the stopper 594. In this instance, with the saline bag coupled to the radioembolization administration set 540 via the contrast line 10B and the contrast port 556B, saline from the saline bag is pulled into the internal chamber 588 of the vial body 589 through the proximal manifold 555B and the needle 559. Accordingly, with the vial body 589 being preloaded with a radioactive fluid media (e.g., radioembolizing microspheres), the saline is effectively mixed with the radioactive fluid media within the vial body 589 as the plunger 584 is retracted from the internal chamber 588 and the negative pressure is generated through the delivery device 500.

The radioembolization administration set 540 further includes one-way check valves 553A in-line with the contrast line 10B and the flushing line 10C. In particular, the one-way check valves 553A are configured to permit fluid communication from the contrast port 556B and the flushing port 556C into the manifolds 555A, 555B, and further configured to prevent fluid communication from the manifolds 555A, 555B to the contrast port 556B and the flushing port 556C. Accordingly, it should be understood that the dose delivered from the vial body 589 to the manifold 555A, 555B is incapable of being directed into the contrast line 10B or the flushing line 10C due to the one-way check valves 553A positioned therein. Thus, the dose is directed to the dose delivery port 556A and received at the catheter fluidly coupled thereto by the dose delivery line 10A. In other words, the one-way check valves 553A prevent a backflow of fluid into the radioembolization administration set 540 and/or the vial assembly 580 coupled thereto.

### II. Radiation Determination Embodiments

As briefly noted above, FIGS. 5-15, discussed in more detail herein, generally relate to embodiments for radiation delivery determination systems 1000 to assist with determining an amount of radiation within at least a portion of the delivery device 500. For instance, the systems 1000 discussed herein may be employed to determine an amount of radiation within at least a portion of the delivery device 500 following a treatment operation or administration of a radioactive dose of fluid from the delivery device 500. More particularly, the systems 1000 discussed herein may be employed to determine an amount of radiation within the radioembolization administration set 540, including the vial assembly 580 that may be partially received within the radioembolization administration set 540, and the tubing 10 (i.e. the dose delivery line 10A, the contrast line 10B, and the flushing line 10C) coupled to the radioembolization administration set 540. By determining an amount of radiation within at least a portion of the delivery device 500 following an administration of a radioactive dose, and knowing a predetermined amount of radiation, or radioactive fluid media, preloaded within the vial body 589, an amount of radiation administered to a patient can be determined and/or verified.

Current systems and methods may involve placing a radioembolization administration set, vial assembly, and/or tubing loosely in a testing container and measuring an amount of radiation in the radioembolization administration set, vial assembly, and/or tubing within the testing container with a radiation measurement device. However, the specific orientation and position of the radioembolization administration set, vial assembly, and/or tubing within the testing container may not be known, and the distance from the source of radiation and the radiation measurement device may affect the amount of radiation detected by the radiation measurement device. Particularly, the intensity of radiation measured is inversely proportional to the square of the distance between the source of radiation and the radioactive measurement device, such that the intensity of radiation detected decreases with increasing distance between the source of radiation and the radioactive measurement device. For instance, an activity of radiation measured with a radioactive measurement device positioned 0.3 meters from the source of radiation may be 10 millicurie (or an equivalent amount of Becquerel), while the activity of radiation measured with the radioactive measurement device positioned 0.4 meters from the same source of radiation may be 6.25 millicurie (or an equivalent amount of Becquerel). Therefore, users often must take several readings at different locations around the testing container and average the results of the several readings to accurately determine the amount of radiation within the radioembolization administration set, vial assembly, and/or tubing.

Moreover, different materials attenuate radiation to different degrees. Therefore, due to the difference in absorption, or radiation shielding properties, between the materials used to form the radioembolization administration set, vial assembly, and/or tubing, the same amount of radiation in the tubing and radioembolization administration set, for instance, may generate a different readout by the radioactive measurement device. That is, the radioembolization administration set 540 may shield 50% of the radiation within from the radioactive measurement device, and the tubing may shield nearly 0% of the radiation within from the radioactive measurement device. Accordingly, a readout of 10 millicurie (or an equivalent amount of Becquerel) by the radioactive measurement device may relate to an actual radiation activity of 20 millicurie (or an equivalent amount of Becquerel) in the radioembolization administration set and 0 millicurie or Becquerel in the tubing, or an actual radiation activity of 0 millicurie or Becquerel in the radioembolization set and 10 millicurie (or an equivalent amount of Becquerel) in the tubing. Without normalizing, or otherwise accounting for the differences in radiation shielding of the various components of the delivery device, a true amount of radiation within the various components of the radioembolization delivery device (i.e. the radioembolization set, the vial assembly, and/or tubing) may not accurately be determined as a whole. The systems described herein allow for accurate and reproducible radiation measurements.

Referring briefly to FIG. 14, a radiation delivery determination system 1000 (also referable to as system 1000 herein) for determining an amount of radiation within at least a portion of the delivery device 500 is depicted. The system 1000 includes a container 600 and a radioactive measurement device 750 external to the container 600. The container 600 may receive the radioembolization administration set 540, including the vial assembly 580 that may be partially received within the radioembolization administration set 540, and the tubing 10 coupled to the radioembolization administration set 540. In embodiments, as described in greater detail below, the container 600 includes a first compartment 610 defining an internal cut-out 612 sized and shaped to receive a periphery of a radioembolization administration set 540, and a second compartment 620 defining a pocket 622 configured to receive tubing 10. The tubing 10 when received may be in fluid communication with and attached to the radioembolization administration set 540. The radioembolization administration set 540 may include an at least partially administered vial of radioactive therapeutic material from vial assembly 580, and the tubing 10 may include at least a first tube, such as the dose delivery line 10A, for administering radioactive therapeutic material from the vial of vial assembly 580. A casing 800 may house the container 600 therein. The radioactive measurement device 750 external to and spaced from the casing 800 and the container 600 is configured to measure an amount of radiation within the radioembolization administration set 540, including the vial assembly 580 that may be partially received therein, and the tubing 10 coupled to the radioembolization administration set 540.

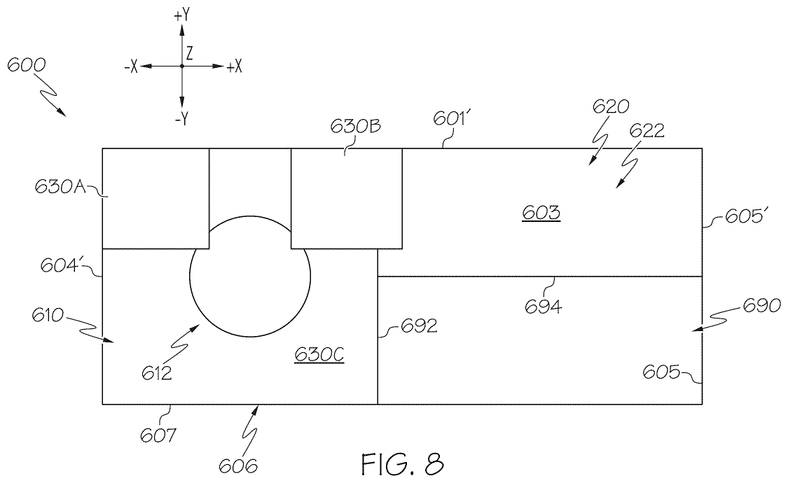

Referring now to FIG. 5, the container 600 is depicted. The container 600 includes a rear surface 601 of a rear wall 601′, a top surface 602 of a top wall 602′, a bottom surface 603 of a bottom wall 603′, a first side surface 604 of a first side wall 604′, and a second side surface 605 of a second side wall 605′. The container also includes a front wall 606 (not depicted in FIG. 5, see FIG. 8), which may be predominantly parallel with the rear surface 601 and orthogonal with the top surface 602, the bottom surface 603, the first side surface 604, and the second side surface 605. While the container 600 is described and depicted as a six-sided structure herein, it should be appreciated that the container 600 may include more or fewer surfaces, so long as the container defines a volume configured to receive the radioembolization administration set 540, which may include the vial assembly 580 that may be partially received therein, and the tubing 10 coupled to the radioembolization administration set 540, as described herein.

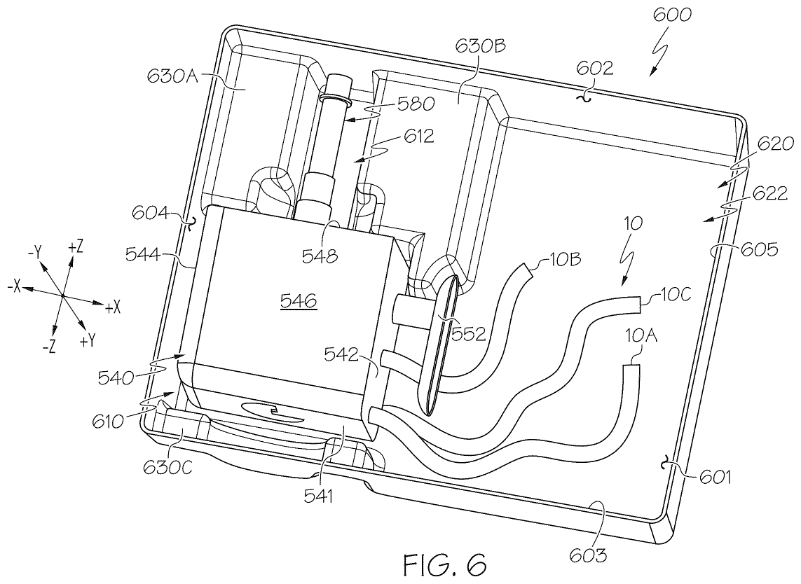

As described above, the container 600 includes a first compartment 610 and a second compartment 620. The first compartment 610 includes an internal cut-out 612 sized and shaped to receive a periphery of the radioembolization administration set 540 as shown in FIG. 6. As used herein, the periphery of the radioembolization administration set 540 may be defined by the proximal end 544, the distal end 542, the pair of sidewalls 546, the top surface 548, the bottom surface 541, the handle 552, and any protrusions, contours, or surface features therein. The internal cut-out 612 of the first compartment 610 is further sized and shaped to receive a periphery of the vial assembly 580 that may be partially received in the radioembolization administration set 540.

Referring again to FIG. 5, the first compartment 610 may be partially defined by portions of the rear surface 601, the top surface 602, the bottom surface 603, the first side surface 604, and the front surface (not depicted). The first compartment 610 may be further defined by one or more internal projections 630A, 630B, 630C. The internal projections 630A, 630B, 630C may be integral with and extend inwardly from one or more of the rear surface 601, the top surface 602, the bottom surface 603, the first side surface 604, the second side surface 605, and/or the front surface (not depicted) of the container 600. At least portions of the internal projections 630A, 630B, 630C, along with the rear surface 601, the top surface 602, the bottom surface 603, the first side surface 604, and the front surface (not depicted) may define the internal cut-out 612. The internal projections 630A, 630B, 630C may be at least partially molded to correspond to at least a portion of the periphery of the radioembolization administration set 540. In being shaped to receive the periphery of the radioembolization administration set 540, the internal cut-out 612 secures the radioembolization administration set 540 within the first compartment 610 such that movement of the radioembolization administration set 540 is limited or eliminated. Moreover, the internal cut-out 612 may be sized and shaped such that the radioembolization administration set 540 is positionable therein in a single orientation.

The second compartment 620 defines a pocket 622 configured to receive the tubing 10 (i.e. the dose delivery line 10A, contrast line 10B, and flushing line 10C) coupled to the radioembolization administration set 540 as shown in FIG. 6. The second compartment 620 may be partially defined by portions of the rear surface 601, the top surface 602, the bottom surface 603, the second side surface 605, and the front surface (not depicted). The second compartment 620 may further be defined by portions of the internal projections 630B and 630C, for instance. That is, and with specific reference to the internal projection 630B, a first surface of the internal projection 630B may at least partially define the first compartment 610, and a second surface of the internal projection 630B may at least partially define the second compartment 620.

The container 600 may include one or more adjustable walls to allow a user access to the first compartment 610 and the second compartment 620. For instance, and without limitation, the front wall 606 (see FIG. 8) may be an adjustable wall that is hingedly secured to the first side wall 604′ and selectively secured to the second side wall 605′ by a securement mechanism such as a latch, interference fit, snap fit assembly, and/or other suitable securement features. In embodiments, the one or more adjustable walls may be removable from the container 600. For instance, and without limitation, the front wall 606 may be selectively secured to both the first side wall 604′ and the second side wall 605′ by a securement mechanism such as a latch, interference fit, snap fit assembly, and/or other suitable securement features. The front wall 606 defining a front surface as a measurement-facing surface 607 may be selectively secured to walls 602′, 603′ defining the top surface 602 and the bottom surface 603 instead of or in addition to the walls 604′, 605′ defining first side surface 604 and the second side surface 605. It should further be appreciated that walls defining any of the rear surface 601, top surface 602, bottom surface 603, first side surface 604, and second side surface 605 may be adjustable, such as through a hinged securement to another surface or wall, instead of or in addition to the front wall 606. Therefore, in embodiments, the user may gain access to the first compartment 610 and the second compartment 620 from the top, bottom, front, rear, or side walls. When the container 600 is “closed,” that is, when the one or more adjustable wall of the container 600 is secured along all of its edges to one or more other walls of the container 600, the container 600 may be leak-tight. Therefore, any liquid radioactive material within the container 600 may be maintained within the boundaries of the container 600.

In embodiments, the container 600 may be transparent such that the interior of the container 600 is viewable through a wall of the container 600. In embodiments, the container 600 may be made of polycarbonate, polymethyl methacrylate, polyethylene terephthalate, polyethylene terephthalate glycol, polyvinyl chloride, cyclic olefin copolymers, polyethylene, polypropylene, styrene methyl methacrylate, styrene acrylonitrile resin, polystyrene, and/or methyl methacrylate acrylonitrile butadiene styrene. The container 600 may be disposable. Therefore, the container 600 may be used once to determine the radioactive content of an individual radioembolization administration set 540 (along with the tubing 10 coupled thereto and vial assembly 580 partially received therein) and discarded. In other words, the container 600 may, optionally, not be re-usable. This allows a user to safely dispose of a container 600 contaminated with radioactive material. This further also allows a user to select a clean, non-contaminated container 600 for each measurement of radioactive material in a radioembolization administration set 540 (along with the tubing 10 coupled thereto and vial assembly 580 partially received therein), ensuring that a potentially radioactively contaminated container 600 does not compromise future radioactive measurements.

Referring now to FIG. 6, the container 600 including the radioembolization administration set 540 and the tubing 10 (i.e., the dose delivery line 10A, the contrast line 10B, and the flushing line 10C) coupled thereto is depicted. The radioembolization administration set 540 and the vial assembly 580 partially received therein are disposed within the internal cut-out 612 of the first compartment 610 of the container 600. The tubing 10 coupled to the radioembolization administration set 540 is disposed within the pocket 622 of the second compartment 620 of the container 600. As discussed above, the internal cut-out 612 limits or eliminates movement of the radioembolization administration set 540 and accepts the radioembolization administration set 540 in a single orientation in a secured fit. Therefore, the container 600 allows for reproducible measurement procedures. That is, when the radioembolization administration set 540 and the vial assembly 580 partially received therein are disposed within the internal cut-out 612, the precise position of the radioembolization administration set 540 and the vial assembly 580 partially received therein in relation to a measurement-facing surface 607 (see FIG. 8) of the container 600 and/or a radioactive measurement device may be determined. As used herein, the measurement-facing surface 607 of the container 600 refers to the surface of the container 600 that is nearest to and facing a radioactive measurement device. In other words, the amount of radiation within the container 600 is measured “through” the measurement-facing surface 607. The container 600, and the internal cut-out 612 of the first compartment 610, therefore, reduces the geometric variability of the radioembolization administration set 540 and vial assembly 580 partially received therein and the impacts of such variability on the readouts from the radioactive measurement device.

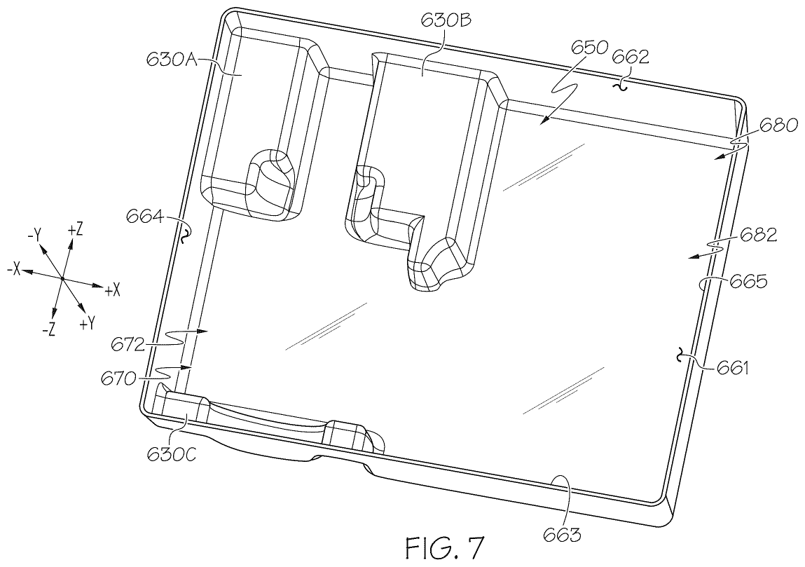

In some embodiments, the internal projections 630A, 630B, 630C within the container 600 may not be integral with one or more of the rear surface 601, the top surface 602, the bottom surface 603, the first side surface 604, the second side surface 605, and/or the front surface (not depicted) of the container 600. For instance, and with reference to FIG. 7, the one or more internal projections 630A, 630B, 630C may be formed in a surface of a liner 650. The liner 650 may include a rear surface 661, a top surface 662, a bottom surface 663, a first side surface 664, and a second side surface 665, each respectively configured to be disposed against the rear surface 601, the top surface 602, the bottom surface 603, the first side surface 604, and the second side surface 605 of the container 600. The liner 650 may not include a front surface to allow for ease of placement of the radioembolization administration set 540, tubing 10 coupled thereto, and the vial assembly 580 at least partially received therein within the liner 650. In embodiments, the liner 650 may include a front surface, and may not include at least one of the rear surface 661, the top surface 662, the bottom surface 663, the first side surface 664, and the second side surface 665 to allow for ease of placement of the radioembolization administration set 540, tubing 10 coupled thereto, and the vial assembly 580 at least partially received therein within the liner 650.

The liner 650 at least partially defines a first compartment 670, which resembles and is configured similar to the first compartment 610 discussed with respect to FIG. 5 in function and disposed within the first compartment 670, and a second compartment 680, which resembles and is configured similar to the second compartment 620 discussed with respect to FIG. 5 in function and disposed within the second compartment 620. The first compartment 670 at least partially defines an internal cut-out 672, configured similar to the internal cut-out 612 discussed with respect to FIG. 6 in function. The second compartment 680 at least partially defines a pocket 682, configured similar to the pocket 622 discussed with respect to FIG. 5.

Referring to FIG. 7 in conjunction with FIG. 5, the liner 650 may be selectively inserted and removed from the container 600. In operation, a user may place the radioembolization administration set 540, tubing 10 coupled thereto, and the vial assembly 580 at least partially received therein within the liner 650. The user may then move the one or more adjustable walls of the container 600 to gain access to an interior of the container 600 to place the liner 650 therein. The user may then move (such as to close) the one or more adjustable walls of the container 600 to seal the container 600. As noted, when using the liner 650 including internal projections 630A, 630B, 630C, the container 600 may not include any internal projections. Portions of the rear surface 601, the top surface 602, the bottom surface 603, the first side surface 604, the second side surface 605, and the front wall 606 of the container 600 may partially define the first compartment 670, and the internal cut-out 672 defined therein, and the second compartment 680, and the pocket 682 defined therein, along with the liner 650. For instance, in embodiments where the liner 650 does not include a front surface, an inner-facing front surface of the front wall 606 of the container 600 may partially define the first compartment 670 and the second compartment 680 when the liner 650 is inserted in the container 600.

In embodiments, the container 600 and/or the liner 650 may be shaped to maintain the tubing 10 coupled to the radioembolization administration set 540 at a desired position within the pocket 622, 682 of the container 600 and/or liner 650, respectively. For instance, and with reference to FIG. 8, which depicts a top view of the container 600 of FIG. 5 according to some embodiments, the container 600 may limit the volume of the pocket 622, or in other words, maintain the tubing 10 coupled to the radioembolization administration set 540 at a specified position within the pocket 622. For instance, the front wall 606 of the container 600 may include a projection 690, which projects from the front wall 606 inward toward the pocket 622. The projection 690 includes a first surface 692 and a second surface 694. The projection 690 may be defined by a portion of the front wall 606 of the container 600, the first surface 692 of the projection 690, the second surface 694 of the projection 690, and a portion of the second side surface 605 of the second side wall 605′ of the container 600.

The interior of the projection 690 may be solid. That is, the area bounded by the front wall 606 of the container 600, the first surface 692 of the projection 690, the second surface 694 of the projection 690, and the second side surface 605 of the container 600 may be a solid material. In embodiments, the first surface 692 of the projection 690, the second surface 694 of the projection 690, and the interior of the projection 690 may be the same material. In embodiments, the first surface 692 of the projection 690, the second surface 694 of the projection 690, and the interior of the projection 690 may be formed of different materials. In embodiments, the first surface 692 of the projection 690, the second surface 694 of the projection 690, and the interior of the projection 690 may be formed of the same material as the front wall 606, or other surface, of the container 600.

Alternatively, the interior of the projection 690 may be hollow. That is, the area bounded by the front wall 606 of the container 600, the first surface 692 of the projection 690, the second surface 694 of the projection 690, and the second side surface 605 of the container 600 may be empty. In embodiments, the first surface 692 of the projection 690 and the second surface 694 of the projection 690 may be the same or different materials. In embodiments, the first surface 692 of the projection 690 and the second surface 694 of the projection 690, may be formed of the same material as the front wall 606, or other surface, of the container 600.

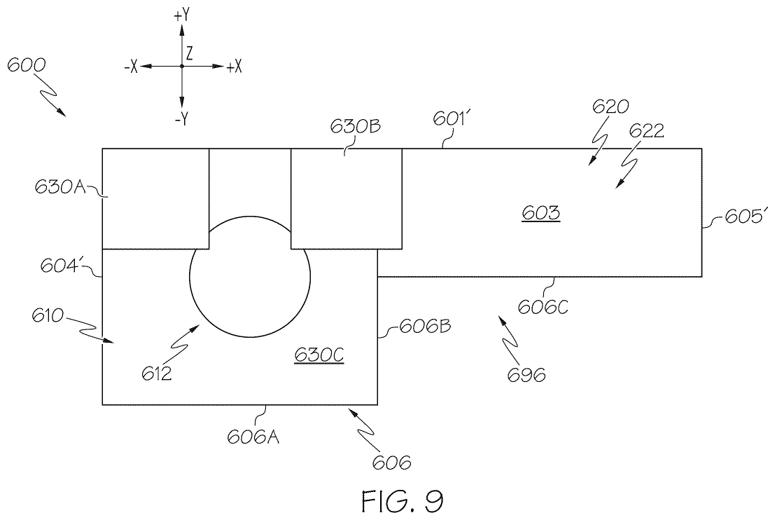

In other embodiments, and with reference to FIG. 9, which depicts a top view of the container 600 of FIG. 5, the container 600 may include a contour 696 to maintain the tubing 10 coupled to the radioembolization administration set 540 at a desired position within the pocket 622 of the container 600. The contour 696 may be formed in the front wall 606 of the container 600. For instance, the front wall 606 may include a first portion 606A of the front wall 606, a second portion 606B of the front wall 606, and a third portion 606C of the front wall 606. The first portion 606A of the front wall 606 may at least partially define the internal cut-out 612. The third portion 606C of the front wall 606 may at least partially define the pocket 622. The second portion 606B of the front wall 606 may join the first portion 606A and the third portion 606C of the front wall 606. The third portion 606C of the front wall 606 may be offset from the first portion 606A of the front wall 606 in the direction of the y-axis of the coordinate axes of FIG. 9, for instance, thereby forming a portion of the contour 696. The contour 696, and more specifically, the third portion 606C of the front wall 606 limits the volume of the pocket 622, or in other words, maintains the tubing 10 coupled to the radioembolization administration set 540 at a specified position within the pocket 622.

While the projection 690 and contour 696 discussed above have been described as formed in or from the front wall 606 of the container 600, the projection 690 and contour 696 may be similarly formed in or from other surfaces or walls such as the rear wall 601′ of the container 600. Forming the projection 690 and/or contour 696 in the front wall 606 and/or rear wall 601′ may be particularly advantageous when the front wall 606 or rear wall 601′ includes the measurement-facing surface of the container 600. That is, the projection 690 and/or contour 696, when formed in or from the front wall 606 and/or rear wall 601′, may maintain the tubing 10 in the pocket 622 a known distance from a radioactive measurement device, in the direction of measurement, when the front wall 606 or rear wall 601′ includes the measurement-facing surface.

The projection 690 and/or contour 696 may also be formed in the top wall 602′ (FIG. 5) and/or bottom wall 603′ of the container 600. Forming the projection 690 and/or contour 696 in the top wall 602′ (FIG. 5) or bottom wall 603′ may be particularly advantageous when the top wall 602′ (FIG. 5) or bottom wall 603′ includes the measurement-facing surface of the container 600. That is, the projection 690 and/or contour 696, when formed in or from the top wall 602′ (FIG. 5) and/or bottom wall 603′, may maintain the tubing 10 in the pocket 622 a known distance from a radioactive measurement device, in the direction of measurement, when the top wall 602′ (FIG. 5) or bottom wall 603′ is the measurement-facing surface. The projection 690 and/or contour 696 may also be formed in the second side wall 605′ of the container 600. Forming the projection 690 and/or contour 696 in the second side wall 605′ may be particularly advantageous when the second side wall 605′ or first side wall 604′ includes the measurement-facing surface of the container 600. That is, the projection 690 and/or contour 696, when formed in or from the second side wall 605′, may maintain the tubing 10 in the pocket 622 a known distance from a radioactive measurement device, in the direction of measurement, when the first side wall 604′ or second side wall 605′ includes the measurement-facing surface of the container 600.

Referring to FIGS. 5 and 7, while the pockets 622 and 682 have been described herein as specifically containing the tubing coupled to the radioembolization administration set 540, it should be appreciated that the pockets 622 and 682 may include other objects instead of or in addition to the tubing. For instance, after a treatment operation, a user may place her gloves in the pocket 622 or 682, which may or may not be contaminated with radioactive material. As another example, a user may place a towel used to absorb a spill or leakage of radioactive material in the pockets 622 or 682. Generally, following a treatment operation, any item that may include or be contaminated with radioactive material and possesses little to no radioactive shielding properties, may be placed in the pocket 622 or 682 along with the tubing.

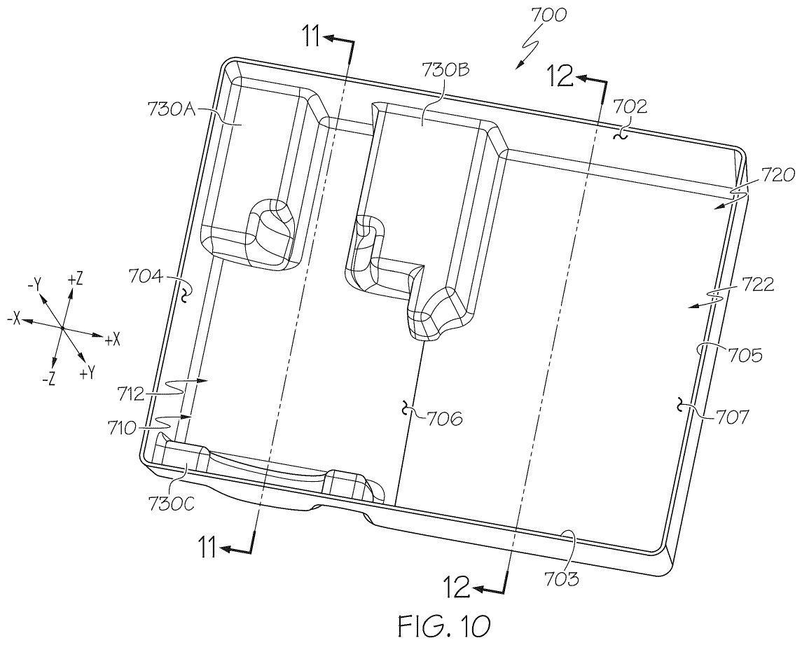

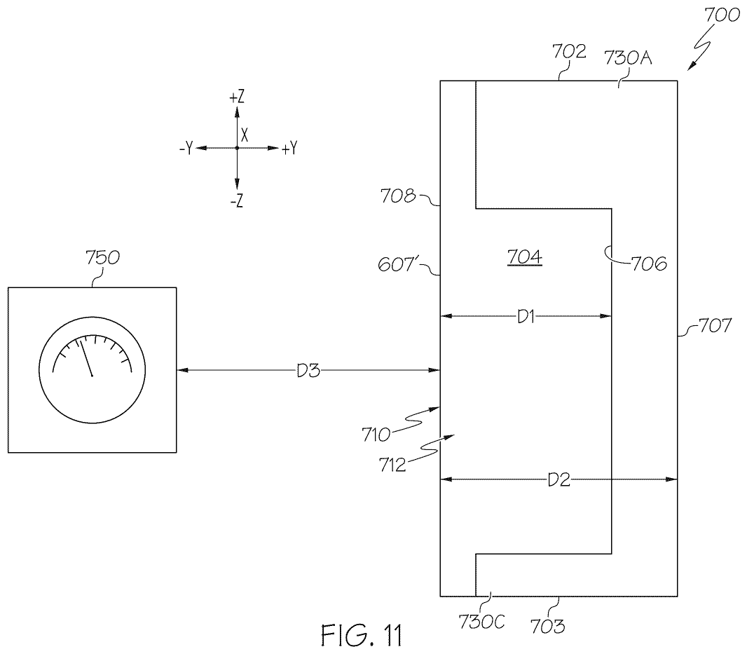

Referring now to FIG. 10, another container 700 is depicted. The container 700 may resemble the container 600 discussed with reference to FIGS. 5 and 6 except as noted herein, such as with respect to offset surfaces 706, 707. The container 700 includes a top surface 702, a bottom surface 703, a first side surface 704, and a second side surface 705. The container also includes a rear first surface 706 and a rear second surface 707. The container also includes a front wall 708 (see FIG. 11), which may be predominantly parallel with the rear first surface 706 and the rear second surface 707 and orthogonal with the top surface 702, the bottom surface 703, the first side surface 704, and the second side surface 705.

The container 700 includes a first compartment 710 and a second compartment 720. The first compartment 710 includes an internal cut-out 712 sized and shaped to receive a periphery of the radioembolization administration set 540. The internal cut-out 712 of the first compartment 710 is further sized and shaped to receive a periphery of the vial assembly 580 that may be partially received in the radioembolization administration set 540. The first compartment 710 may be partially defined by the rear first surface 706, the top surface 702, the bottom surface 703, the first side surface 704, and the front wall 708 (see FIG. 11). The first compartment 710 may be further defined by one or more internal projections 730A, 730B, 730C. The internal projections 730A, 730B, 730C, along with the rear first surface 706, the top surface 702, the bottom surface 703, the first side surface 704, and the front wall 708 (see FIG. 11) define the internal cut-out 712.

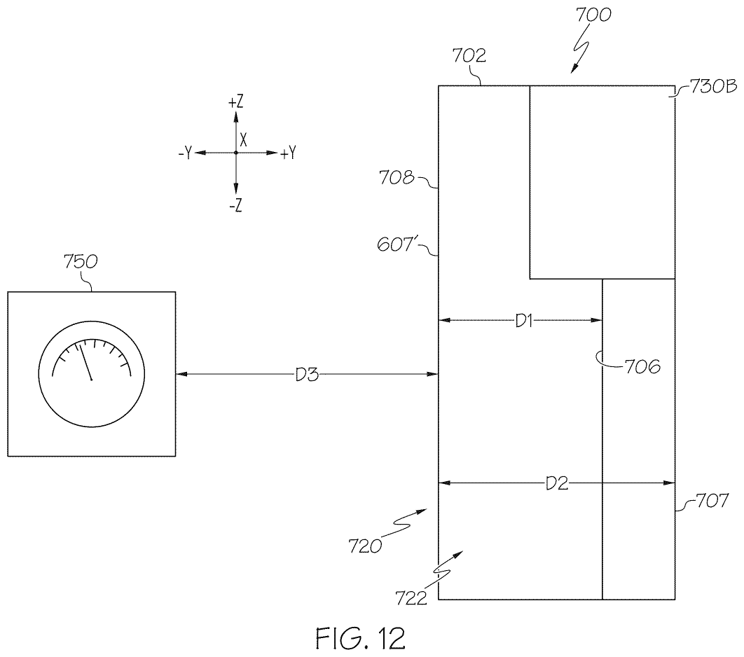

The second compartment 720 defines a pocket 722 configured to receive the tubing 10 (i.e. the dose delivery line 10A, contrast line 10B, and flushing line 10C) coupled to the radioembolization administration set 540. The second compartment 720 and the pocket 722 may be partially defined by the rear second surface 707, the top surface 702, the bottom surface 703, the second side surface 705, and the front wall 708 (see FIG. 12). The second compartment 720 may further be defined by the internal projections 730B and 730C, for instance. That is, and with specific reference to the internal projection 730B, a first surface of the internal projection 730B may at least partially define the first compartment 710, and a second surface of the internal projection 730B may at least partially define the second compartment 720.

Referring now to FIGS. 11 and 12, cross-sectional views of the container 700 about lines 11-11 and 12-12, respectively, of FIG. 10 are depicted. Specifically, FIG. 11 depicts a cross-sectional view of the internal cut-out 712 of the first compartment 710, and FIG. 12 depicts a cross-sectional view of the pocket 722 of the second compartment 720. The container 700 includes a measurement-facing surface 607′. For instance, the front wall 708 defines the measurement-facing surface 607′ of the container 700. Therefore, the measurement-facing surface 607′ of the front wall 708 of the container 700 is nearest to and facing the radioactive measurement device 750, and the rear first surface 706 and the rear second surface 707 are disposed opposite the measurement-facing surface 607′. The radioactive measurement device 750 is external to the container 700 and configured to measure an amount of radiation within the radioembolization administration set 540, the vial assembly 580 that may be positioned therein, and the tubing 10 coupled to the radioembolization administration set 540. The front wall 708, and in the embodiment depicted, the measurement-facing surface 607′, is a distance D3 from the radioactive measurement device 750. The rear first surface 706 defining the internal cut-out 712 of the first compartment 710 is disposed opposite the measurement-facing surface 607′ of the container 700 and is spaced a first distance D1 from the measurement-facing surface 607′ of the container 700, and the rear second surface 707 defining the pocket 722 of the second compartment 720 is disposed opposite the measurement-facing surface 607′ of the container 700 and is spaced a second distance D2 from the measurement-facing surface 607′ of the container 700, wherein the second distance D2 is greater than the first distance D1. For instance, the rear first surface 706 is a distance D1 from the front wall 708. The rear second surface 707 is a distance D2, which is greater than D1, from the front wall 708. In embodiments, as depicted, the rear second surface 707 is the absolute rear surface of the container 700. In other embodiments, the rear second surface 707 may be offset a distance from the absolute rear surface of the container 700. In such embodiments, the rear first surface 706 is a distance D1 from the front wall 708, the rear second surface 707 is a distance D2, which is greater than D1, from the front wall 708, and the absolute rear surface of the container 700 is a distance greater than D2 from the front wall 708.

The rear first surface 706, being closer to the front wall 708, and therefore the radioactive measurement device 750, than the rear second surface 707 effectively positions the radioembolization administration set 540, including the vial assembly 580 that may be partially received therein, closer to the radioactive measurement device 750 than the tubing 10 coupled to the radioembolization administration set 540. Notably, and as discussed above, the radioembolization administration set 540 and/or the vial assembly 580 partially received therein may include materials particularly selected to shield a user from the radioactive material therein. The tubing 10 coupled to the radioembolization administration set 540 may display a reduced amount of shielding when compared to the radioembolization administration set 540 and/or the vial assembly 580. Therefore, the components exhibiting increased absorption or shielding are positioned within the internal cut-out 712 and maintained at a closer distance to the radioactive measurement device 750 than the components (i.e. the tubing 10) exhibiting a lesser amount of absorption, which are positioned within the pocket 722.

As discussed above, the intensity of radiation measured, or the amount of radiation exposed to the radioactive measurement device 750, increases with a decreasing distance between a source of radiation and the radioactive measurement device 750. Therefore, the combined reduction in radiation emission intensity caused by the shielding properties of the materials of the radioembolization administration set 540 and/or vial assembly 580 and the distance between the radioembolization administration set 540 and vial assembly 580 that may be partially received therein and the radioactive measurement device 750 may be equal to the combined reduction in radiation emission intensity by the shielding property of the materials of the tubing 10 and the distance between the tubing 10 and the radioactive measurement device 750. In other words, the radiation emission intensity of all radioactive material within the container 700 may be, regardless of whether contained in the radioembolization administration set 540, vial assembly 580, or tubing 10, reduced to the same degree. That is, the difference in emission intensity reduction caused by the material properties of the radioembolization administration set 540 and/or vial assembly 580 and the tubing 10 may be offset by the difference in emission intensity reduction caused by the distance between the radioembolization administration set 540 and/or vial assembly 580 and the radioactive measurement device 750 and the distance between the tubing 10 and the radioactive measurement device 750.

It should be appreciated that in embodiments including an insertable liner within a container, such as the liner 650 of FIG. 7, the rear surface 661 of the liner 650 may include a first rear surface that partially defines the first compartment 670 and a second rear surface that partially defines the second compartment 680. The rear first surface of the liner 650 may be a first distance from a measurement-facing surface of the container the liner 650 is inserted in, and the rear second surface of the liner 650 may be a second distance, which is greater than the first distance, from the measurement-facing surface of the container the liner 650 is inserted in. In embodiments, the measurement-facing surface of the container may be part of the front wall, such that the first rear surface of the liner 650 and the second rear surface of the liner 650 are disposed on a wall opposite the wall of the measurement-facing surface of the container.

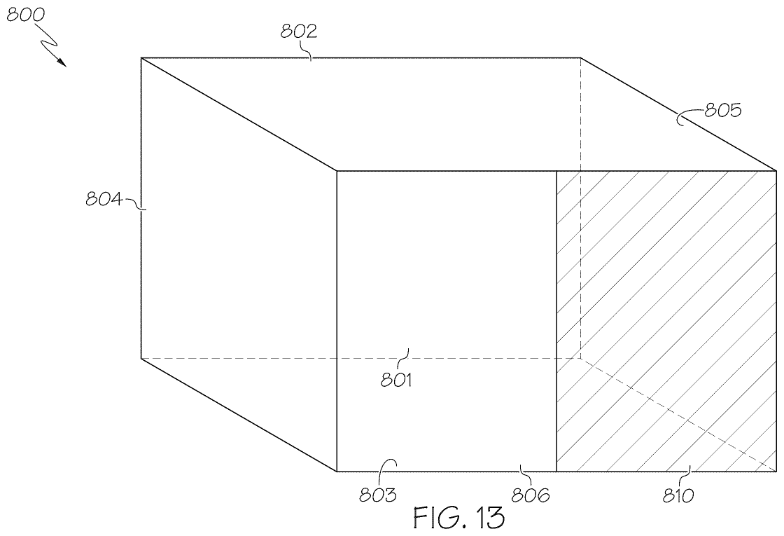

Referring now to FIG. 13, the casing 800 is depicted that is configured to house any of the containers 600, 700 as described herein. The casing 800 may include a rear wall 801, a top wall 802, a bottom wall 803, a first side wall 804, a second side wall 805, and a front wall 806. While the casing 800 is described and depicted as a six-sided structure herein, it should be appreciated that the casing 800 may include more or fewer surfaces or walls, so long as the casing defines a volume able to receive a container according to any of the above-described embodiments.

The casing 800 may include one or more adjustable walls that are configured to close or open to allow a user access to the interior of the casing 800. For instance, and without limitation, the top wall 802 may be hingedly secured to the first side wall 804 and selectively secured to the second side wall 805 by means of a latch, interference fit, snap fit assembly, and/or the like. In embodiments, the one or adjustable walls may be removable from the casing 800. For instance, and without limitation, the top wall 802 may be selectively secured to both the first side wall 804 and the second side wall 805 by a securement mechanism such as a latch, interference fit, snap fit assembly, and/or the like. The top wall 802 may be selectively secured to the front wall 806 and the rear wall 801 instead of or in addition to the first side wall 804 and the second side wall 805. It should further be appreciated that any of the rear wall 801, bottom wall 803, first side wall 804, second side wall 805, and front wall 806 may be adjustable, such as through a hinged securement, to another surface or wall, instead of or in addition to the top wall 802. Therefore, in embodiments, the user may gain access to or close off access to the interior of the casing 800 from the top, bottom, front, rear, or side walls. More particularly, a user may place a container including the radioembolization administration set 540, along with the vial assembly 580 that may be partially received therein, and the tubing 10 coupled to the radioembolization administration set 540 (e.g. the container 600 depicted in FIG. 6) within the casing 800.

The casing 800 may be transparent such that the interior of the casing 800 may be viewable through a wall of the casing 800. In embodiments, the container 600, 700 may be transparent. The casing 800 may be made of acrylic. The casing 800 may also be made of polycarbonate, polyethylene terephthalate, polyethylene terephthalate glycol, polyvinyl chloride, cyclic olefin copolymers, polyethylene, polypropylene, styrene methyl methacrylate, styrene acrylonitrile resin, polystyrene, and/or methyl methacrylate acrylonitrile butadiene styrene.

The casing 800 may include a radioactive shield 810 partially extending along the casing 800. The radioactive shield 810 is formed of a material that is configured to inhibit radioactive emissions from radioactive material within the casing 800, and more particularly within the container (e.g. container 600 of FIG. 6) contained therein. The radioactive shield 810 may be a metal. The radioactive shield 810 may include any of stainless steel, lead, tin, copper, pewter, and aluminum. The radioactive shield 810 may extend along any of the rear wall 801, the top wall 802, the bottom wall 803, the first side wall 804, the second side wall 805, and the front wall 806. Generally, the radioactive shield 810 extends along the measurement-facing surface of the casing 800. The measurement-facing surface of the casing 800 is the surface of the casing 800 nearest to and facing a radioactive measurement device. Therefore, it can be said that the measurement of radiation within the casing 800 is “taken through” the measurement-facing surface of the casing 800. In embodiments, the radioactive shield 810 may be integral with the casing 800 such that the radioactive shield 810 forms part of the surface of the casing 800 that the radioactive shield 810 extends along. In embodiments, the radioactive shield 810 may be fixed to the surface of the casing 800 that the radioactive shield 810 extends along by any suitable adhesive or fixation means.

With reference to FIGS. 5, 6, 13, and 14, the radioactive shield 810 extends along the casing 800 such that the second compartment 620 of the container 600 is covered by the radioactive shield 810 of the casing 800, and the first compartment 610 of the container 600 is uncovered by the radioactive shield 810 of the casing 800. Thus, the radioactive shield 810 may be located such that radiation from the second compartment 620 to the radioactive measurement device passes through the radioactive shield 810 of the casing 800, and radiation from the first compartment 610 of the container 600 to the radioactive measurement device does not pass through the radioactive shield 810 of the casing 800. Therefore, and as particularly depicted in FIG. 14, the radioembolization administration set 540, including the vial assembly 580 that may be partially received therein, are uncovered by the radioactive shield 810, and the tubing 10 coupled to the radioembolization administration set 540 is covered by the radioactive shield 810. Notably, and as discussed above, the radioembolization administration set 540 and/or the vial assembly 580 partially received therein may include materials particularly selected to shield a user from the radioactive material therein. The tubing 10 coupled to the radioembolization administration set 540 may display a reduced amount of shielding when compared to the radioembolization administration set 540 and/or the vial assembly 580. Therefore, the components exhibiting increased absorption or shielding are uncovered by the radioactive shield 810, and the components exhibiting a lesser amount of absorption are covered by the radioactive shield 810. Particularly, the radioactive shield 810 may be selected such that all radioactive material within the container 600 may be, regardless of whether contained in the radioembolization administration set 540, vial assembly 580, or tubing 10, shielded to the same degree. That is, the difference in reduction of radiation emission intensity caused by the material properties of the radioembolization administration set 540 and/or vial assembly 580 and the material properties of the tubing 10 may be offset by the radioactive shield 810 covering the tubing 10. In other words, radiation emitted from the second compartment 620 to the radioactive measurement device 750 must pass through the radioactive shield 810 of the casing 800, and radiation emitted from the first compartment 610 to the radioactive measurement device 750 does not pass through the radioactive shield 810 of the casing 800.

Referring now to FIGS. 5, 6, 14 and 15, a method 900 for measuring an amount of radiation within a radioembolization administration set 540 and tubing 10 in fluid communication with the radioembolization administration set 540 will be described. At a block 902 of the method 900, a user removes the radioembolization administration set 540, which may include the vial assembly 580 partially received therein, and the tubing 10 coupled to the radioembolization administration set 540 from the delivery device 500 (See FIG. 1). Particularly, the radioembolization administration set 540, which may include the vial assembly 580 partially received therein, and the tubing 10 coupled to the radioembolization administration set 540 are removed from the delivery device 500 following a treatment operation, or an administration of at least some of an initial radioactive dose preloaded within the vial assembly 580.

At a block 904 of the method 900, a user places the radioembolization administration set 540, which may include the vial assembly 580 partially received therein, and the tubing 10 coupled to the radioembolization administration set 540 in the container 600 (or other containers described herein). More particularly, the radioembolization administration set 540, which may include the vial assembly 580 partially received therein, is placed in the internal cut-out 612 of the first compartment 610, and the tubing 10 coupled to the radioembolization administration set 540 is placed in the pocket 622 of the second compartment 620. As discussed above, the internal cut-out 612 is sized and shaped to receive a periphery of the radioembolization administration set 540, and the vial assembly 580 that may be partially received therein, such that the radioembolization administration set 540, and vial assembly 580 that may be partially received therein, can be reproducibly placed within the container 600.

It should be appreciated that the radioembolization administration set 540, which may include the vial assembly 580 partially received therein, and the tubing 10 coupled to the radioembolization administration set 540 may be placed in any of the above-described containers. That is, in embodiments, the container 600 may optionally include the projection 690 (see FIG. 8) or the contour 696 (see FIG. 9). Moreover, in embodiments, the radioembolization administration set 540, which may include the vial assembly 580 partially received therein, and the tubing 10 coupled to the radioembolization administration set 540 may be placed in the container 700 (see FIGS. 10-12). In embodiments, the user may first place the radioembolization administration set 540, which may include the vial assembly 580 partially received therein, and the tubing 10 coupled to the radioembolization administration set 540 in the liner 650 (see FIG. 7), and then place the liner 650 in the container 600.

At a block 906 of the method 900, a user may place the container 600 in the casing 800. As discussed above, the casing 800 may include a radioactive shield 810 partially extending along the casing 800 such that the second compartment 620 of the container 600 is covered by the radioactive shield 810, and the first compartment 610 of the container 600 is uncovered by the radioactive shield 810.

At a block 908 of the method 900, a user may measure the amount of radiation within the radioembolization administration set 540, which may include the vial assembly 580 partially received therein, and the tubing 10 coupled to the radioembolization administration set 540 with the radioactive measurement device 750. The radioactive measurement device 750 may be any device suitable for detecting and measuring radiation. The radioactive measurement device 750 may be a Geiger counter, an ionization chamber, a proportional counter, a gas-filled detector, a scintillation counter, a semiconductor detector, or other suitable device. The radioactive measurement device 750 may be disposed external and spaced from the container 600 and the casing 800. The radioactive measurement device 750 is configured to measure the amount of radiation within the radioembolization administration set 540, which may include the vial assembly 580 partially received therein, and the tubing 10 coupled to the radioembolization administration set 540 with the radioactive measurement device 750 “through” the measurement-facing surfaces of the container 600 and casing 800. The radioactive measurement device 750 is particularly configured to determine a single measurement for the total amount of radiation within the radioembolization administration set 540, which may include the vial assembly 580 partially received therein, and the tubing 10 coupled to the radioembolization administration set 540. The units of the amount of radiation within the radioembolization administration set 540, which may include the vial assembly 580 partially received therein, and the tubing 10 coupled to the radioembolization administration set 540, measured by the radioactive measurement device 750 may be curie, Becquerel, counts per minute, rem per hour, Sieverts per hour, etc. depending on the particular radioactive measurement device 750 employed.

As noted above, the difference in material properties of the radioembolization administration set 540 and/or vial assembly 580 contained therein and the material properties of the tubing 10 may result in different degrees of shielding or attenuation of the radiation within the radioembolization administration set 540, and/or vial assembly 580 contained therein, and the tubing 10. Therefore, with traditional systems and methods, a measurement of the total radiation within the radioembolization administration set 540, and vial assembly 580 that may be contained therein, and the tubing 10 may be inaccurate. Moreover, as noted above, with traditional systems and methods, a measurement of the total radiation within the radioembolization administration set 540, and vial assembly 580 that may be contained therein, and the tubing 10 may be inaccurate due to the variable placement of the components within a measuring apparatus and the different distances between the various components and the radioactive measurement device 750.

The system 1000 and method 900 described herein address the above challenges and allow for accurate measurement of the total amount of radiation within radioembolization administration set 540, including vial assembly 580 that may be contained therein, and the tubing 10 coupled to the radioembolization administration set 540. Particularly, the above-described containers (e.g. the container 600) may allow for precise and reproducible placement of the radioembolization administration set 540, including vial assembly 580 that may be contained therein, and the tubing 10 coupled to the radioembolization administration set 540. Moreover, the difference in reduction of radiation emission intensity caused by the material properties of the radioembolization administration set 540 and/or vial assembly 580 contained therein and the material properties of the tubing 10 may be offset by the positioning of the of the radioembolization administration set 540, including vial assembly 580 that may be contained therein, and the tubing 10 coupled to the radioembolization administration set 540 and/or the radioactive shield 810 of the casing 800. That is, to reduce the radiation emission intensity of the radiation in the tubing 10 to the same degree as the radiation in the radioembolization administration set 540, including the vial assembly 580 that may be partially received therein, the tubing 10 may be positioned within the container (e.g. the container 600) a known further distance from the radioactive measurement device 750 than the radioembolization administration set 540, and/or the radioactive shield 810 may be positioned between the tubing 10 and the radioactive measurement device 750.

That is, in some embodiments, the greater distance between the tubing 10 and the radioactive measurement device 750 compared to the radioembolization administration set 540, and vial assembly 580 that may be positioned therein, and the shielding of the tubing 10 provided by the radioactive shield 810 may, in combination, reduce the emission intensity of the radiation within the tubing 10 to the same degree as the radiation within the radioembolization administration set 540 and vial assembly 580 that may be partially received therein.

In other embodiments, the greater distance between the tubing 10 and the radioactive measurement device 750 compared to the radioembolization administration set 540, and vial assembly 580 that may be positioned therein, and the radioactive measurement device 750 may, alone, be sufficient to reduce the emission intensity of the radiation within the tubing 10 to the same degree as the radiation within the radioembolization administration set 540 and vial assembly 580 that may be partially received therein. In such embodiments, for instance, the radioembolization administration set 540, including the vial assembly 580 that may be partially received therein, and the tubing 10 may be placed in the container 700 (see FIGS. 10-12) and not placed in the casing 800 prior to measurement by the radioactive measurement device 750. Alternatively, in such embodiments, the radioembolization administration set 540, including the vial assembly 580 that may be partially received therein, and the tubing 10 may be placed in the container 700 (see FIGS. 10-12), which may be placed in the casing 800 prior to measurement by the radioactive measurement device 750. However, in such cases, the casing 800 need not include the radioactive shield 810 to further attenuate the radiation within the tubing 10.

In other embodiments, the shielding of the tubing 10 provided by the radioactive shield 810 may, alone, be sufficient to reduce the emission intensity of the radiation within the tubing 10 to the same degree as the radiation within the radioembolization administration set 540 and vial assembly 580 that may be partially received therein. In such embodiments, for instance, the container (e.g. the container 600) that the radioembolization administration set 540, including the vial assembly 580 that may be partially received therein, and the tubing 10 may be placed in may maintain the radioembolization administration set 540, including the vial assembly 580 that may be partially received therein, and the tubing 10 at approximately the same distance from the measurement-facing surface of the container 600. The container 600 may then be placed in the casing 800 with the radioactive shield 810 partially extending along the casing 800 such that the second compartment 620 of the container 600 is covered by the radioactive shield 810, and the first compartment 610 of the container 600 is uncovered by the radioactive shield 810.

Because the systems 1000 and methods 900 herein allow for the equal reduction of the emission intensity of the radiation within the tubing 10 and within the radioembolization administration set 540, and vial assembly 580 that may be partially received therein, a single correction factor can be applied to the raw measurement by the radioactive measurement device 750 to obtain the true amount of radiation within the tubing 10, radioembolization administration set 540, and vial assembly 580. By determining an amount of radiation remaining in the radioembolization administration set 540, including the vial assembly 580 that may be received therein, and the tubing 10 coupled to the radioembolization administration set 540 following a treatment procedure, a user can determine the percentage of the radioactive dose preloaded within the vial body 589 that was, in fact, delivered to the patient. If less than 80% of the preloaded dose was delivered to the patient, it may be determined that an error or misadministration event occurred during the treatment procedure.

Embodiments have been depicted wherein measurements of the amount of radiation in the radioembolization administration set 540 and/or vial assembly 580 partially received therein are “taken through” a sidewall 546 of the radioembolization administration set 540. That is, the measurement-facing surface of the radioembolization administration set 540, or the surface of the radioembolization administration set 540 nearest to and facing the radioactive measurement device 750 has been depicted as the sidewall 546 herein. However, it should be appreciated that this is a non-limiting example. That is, the particular shape of the internal cut-out in any of the above-described containers and/or liners (see e.g. the internal cut-out 612 of the container 600 of FIG. 6) and/or the orientation of any of the above-described containers (see e.g. the container 600 of FIG. 6) when placed in the casing 800 or otherwise positioned for measurement from the radioactive measurement device 750 may be such that the measurement-facing surface of the radioembolization administration set 540 is the top surface 548 of the radioembolization administration set 540. In such embodiments, the effects of the material properties of the radioembolization administration set 540 on the shielding of the radiation within the vial assembly 580 may be reduced by measuring the amount of radiation in the vial assembly 580 through the opening of the vial chamber 558.

### III. Aspects Listing

Embodiments can be described with reference to the following numerical clauses:

Aspect 1. A radiation delivery determination system, comprising: a container, the container comprising: a first compartment defining an internal cut-out sized and shaped to receive a periphery of a radioembolization administration set; and a second compartment defining a pocket configured to receive tubing, wherein the tubing is in fluid communication with and attached to the radioembolization administration set; and a radioactive measurement device external to the container and configured to measure an amount of radiation within the radioembolization administration set and the tubing.

Aspect 2. The radiation delivery determination system of Aspect 1, wherein the container receives the periphery of the radioembolization device and the tubing is disposed within the pocket of the second compartment of the container.

Aspect 3. The radiation delivery determination system of any preceding Aspect, wherein the radioembolization administration set is disposed within the internal cut-out of the first compartment of the container.

Aspect 4. The radiation delivery determination system of any preceding Aspect, wherein: the container further comprises a measurement-facing surface; a rear first surface defining the internal cut-out of the first compartment is disposed opposite the measurement-facing surface of the container and is spaced a first distance from the measurement-facing surface of the container; and a rear second surface defining the pocket of the second compartment is disposed opposite the measurement-facing surface of the container and is spaced a second distance from the measurement-facing surface of the container, wherein the second distance is greater than the first distance.

Aspect 5. The radiation delivery determination system of any preceding Aspect, wherein the measurement-facing surface of the container is nearest to and facing the radioactive measurement device.

Aspect 6. The radiation delivery determination system of any preceding Aspect, further comprising a casing, wherein the casing is configured to house the container.

Aspect 7. The radiation delivery determination system of any preceding Aspect, wherein the casing further comprises a radioactive shield partially extending along the casing.

Aspect 8. The radiation delivery determination system of any preceding Aspect, wherein the radioactive shield is located such that: radiation from the second compartment to the radioactive measurement device passes through the radioactive shield of the casing; and radiation from the first compartment to the radioactive measurement device does not pass through the radioactive shield of the casing.

Aspect 9. The radiation delivery determination system of any preceding Aspect, wherein the casing is transparent such that an interior of the casing is viewable through a wall of the casing, and the container is transparent such that an interior of the container is viewable through a wall of the container.

Aspect 10. The radiation delivery determination system of any preceding Aspect, wherein the radioactive measurement device is a Geiger counter.

Aspect 11. A radiation delivery determination system, comprising: a container, the container comprising: a first compartment defining an internal cut-out sized and shaped to receive a periphery of a radioembolization administration set, wherein: the radioembolization administration set comprises an at least partially administered vial of radioactive therapeutic material; and a second compartment defining a pocket configured to receive tubing, wherein the tubing is in fluid communication with and attached to the radioembolization administration set, wherein: the tubing is disposed within the pocket of the second compartment of the container; and the tubing comprises at least a first tube for administering radioactive therapeutic material from the vial; and a Geiger counter external to the container and configured to measure a total amount of radiation within the radioembolization administration set and the tubing.

Aspect 12. The radiation delivery determination system of Aspect 11, wherein: the container further comprises a measurement-facing surface, wherein the measurement-facing surface of the container is nearest to and facing the Geiger counter; a rear first surface defining the internal cut-out of the first compartment is disposed opposite the measurement-facing surface of the container and is spaced a first distance from the measurement-facing surface of the container; and a rear second surface defining the pocket of the second compartment is disposed opposite the measurement-facing surface of the container and is spaced a second distance from the measurement-facing surface of the container, wherein the second distance is greater than the first distance.

Aspect 13. The radiation delivery determination system of any preceding Aspect, wherein the container is transparent such that an interior of the container is viewable through a wall of the container.

Aspect 14. The radiation delivery determination system of any preceding Aspect, further comprising a casing, wherein: the casing is configured to house the container; the casing further comprises a radioactive shield partially extending along the casing, wherein the radioactive shield is located such that: radiation from the second compartment to the Geiger counter passes through the radioactive shield of the casing; and radiation from the first compartment to the Geiger counter does not pass through the radioactive shield of the casing.

Aspect 15. The radiation delivery determination system of any preceding Aspect, wherein the casing is transparent such that an interior of the casing is viewable through a wall of the casing.

Aspect 16. A method of measuring an amount of radiation within a radioembolization administration set and tubing in fluid communication with the radioembolization administration set, the method comprising: placing the radioembolization administration set and the tubing in a container, the container comprising: a first compartment defining an internal cut-out sized and shaped to receive a periphery of the radioembolization administration set, wherein: the radioembolization administration set is placed within the internal cut-out of the first compartment; a second compartment defining a pocket configured to receive the tubing, wherein the tubing is placed within the pocket of the second compartment; and measuring the amount of radiation within the radioembolization administration set and the tubing with a radioactive measurement device disposed external to and spaced from the container.

Aspect 17. The method of Aspect 16, wherein: the container further comprises a measurement-facing surface, wherein the measurement-facing surface of the container is nearest to and facing the radioactive measurement device; a rear first surface defining the internal cut-out of the first compartment is disposed opposite the measurement-facing surface of the container and is spaced a first distance from the measurement-facing surface of the container; and a rear second surface defining the pocket of the second compartment is disposed opposite the measurement-facing surface of the container and is spaced a second distance from the measurement-facing surface of the container, wherein the second distance is greater than the first distance.

Aspect 18. The method of any preceding Aspect, further comprising placing the container in a casing, the casing comprising a radioactive shield partially extending along the casing, wherein the radioactive shield is located such that: radiation from the second compartment to the radioactive measurement device passes through the radioactive shield of the casing; and radiation from the first compartment of the container to the radioactive measurement device does not pass through the radioactive shield of the casing.

Aspect 19. The method of any preceding Aspect, wherein the radioactive shield comprises at least one of stainless steel, aluminum, or pewter, and the casing comprises an acrylic material.

Aspect 20. The method of any preceding Aspect, wherein the radioactive measurement device is a Geiger counter.

It should now be understood that embodiments of the present disclosure are directed to a radiation delivery determination system for determining an amount of radiation within a radioembolization administration set, including a vial assembly that may be received therein, and tubing coupled to the radioembolization administration set. The radiation delivery determination systems includes a container. The container includes a first compartment defining an internal cut-out sized and shaped to receive a periphery of the radioembolization administration set and a second compartment defining a pocket configured to receive tubing. In embodiments, to normalize a reduction of radiation emission intensity of radiation contained within the radioembolization administration set and/or vial assembly and the tubing, a rear first surface of the first compartment disposed opposite a measurement-facing surface of the container is spaced a first distance from the measurement-facing surface of the container, and a rear second surface of the second compartment disposed opposite the measurement-facing surface of the container is spaced a second distance from the measurement-facing surface of the container, where the second distance is greater than the first distance. The measurement-facing surface of the container is nearest to and facing the radioactive measurement device. In embodiments, to normalize a reduction of radiation emission intensity of radiation contained within the radioembolization administration set and/or vial assembly and the tubing, the container is placed within a casing that includes a radioactive shield that partially extends along the casing and covers the second compartment of the container, but does not cover the first compartment of the container.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

For the purposes of describing and defining the present disclosure it is noted that the term “substantially” is used herein to represent the inherent degree of uncertainty that may be attributed to any quantitative comparison, value, measurement, or other representation. The term “substantially” is used herein also to represent the degree by which a quantitative representation may vary from a stated reference without resulting in a change in the basic function of the subject matter at issue. As such, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something slightly less than exact.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A radiation delivery determination system, comprising:
a container, the container comprising:
a first compartment defining an internal cut-out sized and shaped to receive a periphery of a radioembolization administration set; and
a second compartment defining a pocket configured to receive tubing, wherein the tubing is in fluid communication with and attached to the radioembolization administration set; and
a radioactive measurement device external to the container and configured to measure an amount of radiation within the radioembolization administration set and the tubing.

2. The radiation delivery determination system of claim 1, wherein the container receives the periphery of the radioembolization device and the tubing is disposed within the pocket of the second compartment of the container.

3. The radiation delivery determination system of claim 1, wherein the radioembolization administration set is disposed within the internal cut-out of the first compartment of the container.

4. The radiation delivery determination system of claim 1, wherein:
the container further comprises a measurement-facing surface;
a rear first surface defining the internal cut-out of the first compartment is disposed opposite the measurement-facing surface of the container and is spaced a first distance from the measurement-facing surface of the container; and
a rear second surface defining the pocket of the second compartment is disposed opposite the measurement-facing surface of the container and is spaced a second distance from the measurement-facing surface of the container, wherein the second distance is greater than the first distance.

5. The radiation delivery determination system of claim 4, wherein the measurement-facing surface of the container is nearest to and facing the radioactive measurement device.

6. The radiation delivery determination system of claim 1, further comprising a casing, wherein the casing is configured to house the container.

7. The radiation delivery determination system of claim 6, wherein the casing further comprises a radioactive shield partially extending along the casing.

8. The radiation delivery determination system of claim 7, wherein the radioactive shield is located such that:
radiation from the second compartment to the radioactive measurement device passes through the radioactive shield of the casing; and
radiation from the first compartment to the radioactive measurement device does not pass through the radioactive shield of the casing.

9. The radiation delivery determination system of claim 6, wherein the casing is transparent such that an interior of the casing is viewable through a wall of the casing, and the container is transparent such that an interior of the container is viewable through a wall of the container.

10. The radiation delivery determination system of claim 1, wherein the radioactive measurement device is a Geiger counter.

11. A radiation delivery determination system, comprising:
a container, the container comprising:
a first compartment defining an internal cut-out sized and shaped to receive a periphery of a radioembolization administration set, wherein:
the radioembolization administration set comprises an at least partially administered vial of radioactive therapeutic material; and
a second compartment defining a pocket configured to receive tubing, wherein the tubing is in fluid communication with and attached to the radioembolization administration set, wherein:
the tubing is disposed within the pocket of the second compartment of the container; and
the tubing comprises at least a first tube for administering radioactive therapeutic material from the vial; and
a Geiger counter external to the container and configured to measure a total amount of radiation within the radioembolization administration set and the tubing.

12. The radiation delivery determination system of claim 11, wherein:
the container further comprises a measurement-facing surface, wherein the measurement-facing surface of the container is nearest to and facing the Geiger counter;
a rear first surface defining the internal cut-out of the first compartment is disposed opposite the measurement-facing surface of the container and is spaced a first distance from the measurement-facing surface of the container; and
a rear second surface defining the pocket of the second compartment is disposed opposite the measurement-facing surface of the container and is spaced a second distance from the measurement-facing surface of the container, wherein the second distance is greater than the first distance.

13. The radiation delivery determination system of claim 11, wherein the container is transparent such that an interior of the container is viewable through a wall of the container.

14. The radiation delivery determination system of claim 11, further comprising a casing, wherein:
the casing is configured to house the container;
the casing further comprises a radioactive shield partially extending along the casing, wherein the radioactive shield is located such that:
radiation from the second compartment to the Geiger counter passes through the radioactive shield of the casing; and
radiation from the first compartment to the Geiger counter does not pass through the radioactive shield of the casing.

15. The radiation delivery determination system of claim 14, wherein the casing is transparent such that an interior of the casing is viewable through a wall of the casing.

16. A method of measuring an amount of radiation within a radioembolization administration set and tubing in fluid communication with the radioembolization administration set, the method comprising:
placing the radioembolization administration set and the tubing in a container, the container comprising:
a first compartment defining an internal cut-out sized and shaped to receive a periphery of the radioembolization administration set, wherein:
the radioembolization administration set is placed within the internal cut-out of the first compartment;
a second compartment defining a pocket configured to receive the tubing, wherein the tubing is placed within the pocket of the second compartment; and
measuring the amount of radiation within the radioembolization administration set and the tubing with a radioactive measurement device disposed external to and spaced from the container.

17. The method of claim 16, wherein:
the container further comprises a measurement-facing surface, wherein the measurement-facing surface of the container is nearest to and facing the radioactive measurement device;
a rear first surface defining the internal cut-out of the first compartment is disposed opposite the measurement-facing surface of the container and is spaced a first distance from the measurement-facing surface of the container; and
a rear second surface defining the pocket of the second compartment is disposed opposite the measurement-facing surface of the container and is spaced a second distance from the measurement-facing surface of the container, wherein the second distance is greater than the first distance.

18. The method of claim 16, further comprising placing the container in a casing, the casing comprising a radioactive shield partially extending along the casing, wherein the radioactive shield is located such that:
radiation from the second compartment to the radioactive measurement device passes through the radioactive shield of the casing; and
radiation from the first compartment of the container to the radioactive measurement device does not pass through the radioactive shield of the casing.

19. The method of claim 18, wherein the radioactive shield comprises at least one of stainless steel, aluminum, or pewter, and the casing comprises an acrylic material.

20. The method of claim 16, wherein the radioactive measurement device is a Geiger counter.
